SEQUENCE LISTING (10 sequences)

SEQ ID NO: 1 - DNA, 20 bp, Artificial Sequence
  Description of Artificial Sequence Synthetic primer
ccccttcagccagcatataa

SEQ ID NO: 2 - DNA, 20 bp, Artificial Sequence
  Description of Artificial Sequence Synthetic primer
ttctctgggctgtctggact

SEQ ID NO: 3 - DNA, 23 bp, Artificial Sequence
  Description of Artificial Sequence Synthetic primer
aagtttctctgatggaaaacaca

SEQ ID NO: 4 - DNA, 21 bp, Artificial Sequence
  Description of Artificial Sequence Synthetic primer
ggtggctggaaatttcagtag

SEQ ID NO: 5 - DNA, 20 bp, Artificial Sequence
  Description of Artificial Sequence Synthetic primer
tggagccatgattaggaagg

SEQ ID NO: 6 - DNA, 20 bp, Artificial Sequence
  Description of Artificial Sequence Synthetic primer
taaggaaggaccccatttcc

SEQ ID NO: 7 - DNA, 20 bp, Artificial Sequence
  Description of Artificial Sequence Synthetic primer
tgctgcttcatgttctgtga

SEQ ID NO: 8 - DNA, 20 bp, Artificial Sequence
  Description of Artificial Sequence Synthetic primer
tggtgcattagctttggttg

SEQ ID NO: 9 - DNA, 21 bp, Artificial Sequence
  Description of Artificial Sequence Synthetic primer
aaccacaggaacaaaacctca

SEQ ID NO: 10 - DNA, 20 bp, Artificial Sequence
  Description of Artificial Sequence Synthetic primer
cctcctgttggacatttgga

(19) United States
(12) Patent Application Publication
(10) Pub. No.: US 2020/0131573 A1
(43) Pub. Date: Apr. 30, 2020

(54) GENETIC VARIANTS ASSOCIATED WITH HUMAN-DIRECTED HYPER-SOCIAL BEHAVIOR IN DOMESTIC DOGS

(71) Applicants: The Trustees of Princeton University, Princeton, NJ (US); THE REGENTS OF THE UNIVERISITY OF CALIFORNIA, Los Angeles, CA (US); Oregon State University, Corvallis, OR (US)

(72) Inventors: Bridgett vonHOLDT, Lawrenceville, NJ (US); Monique UDELL, Albany, OR (US); Janet SINSHEIMER, Los Angeles, CA (US)

(73) Assignees: The Trustees of Princeton University, Princeton, NJ (US); THE REGENTS OF THE UNIVERSITY OF CALIFORNIA, Los Angeles, CA (US); Oregon State University, Corvallis, OR (US)

(21) Appl. No.: 16/626,374

(22) PCT Filed: Jun. 29, 2018
(86) PCT No.: PCT/US2018/040344

§ 371 Date: Dec. 24, 2019

Related U.S. Application Data
(60) Provisional application No. 62/527,653, filed on Jun. 30, 2017.

(51) Int. Cl. C12Q 1/6876

(52) U.S. Cl. CPC C12Q 1/6876; C12Q 2600/156; C12Q 2600/124

(57) ABSTRACT

Disclosed herein are structural variants in the Williams-Beuren Syndrome locuse of the dog genome that are associated with hyper-social behavior in dogs relative to wolves, and that are informative regarding the nature of social behavior in dogs. Disclosed also is a commercial test with these loci as indicators along the spectrum of sociality. Methods of breeding dogs to select for dogs having increased sociability are also disclosed.

### CROSS REFERENCE TO RELATED APPLICATIONS

This application claims priority from U.S. Provisional Patent Application Ser. No. 62/527,653, filed Jun. 30, 2017, the content of which is hereby incorporated by reference, in its entirety.

### STATEMENT REGARDING FEDERALLY SPONSORED RESEARCH OR DEVELOPMENT

This invention was made with government support under Grant No. GM086887 awarded by the National Institutes of Health, and Grant Nos. DEB-1245373 and DMS-1264153 awarded by the National Science Foundation. The government has certain rights in the invention.

### BACKGROUND

Although considerable progress has been made in understanding the genetic basis of morphologic traits (e.g., body size, coat color) in dogs and wolves, the genetic basis of their behavioral divergence is poorly understood. While decades of research have focused on the unique relationship between humans and domestic dogs, the role of genetics in shaping canine behavioral evolution remains to be elucidated. Existing hypotheses on the behavioral divergence between dogs and wolves posit that dogs are more adept at social problem solving (1) due to an evolved human-like social cognition (2,3). However, mounting evidence suggests that human-socialized wolves can match or exceed the performance of domestic dogs across these socio-cognitive domains (4). Empirical demonstrations remain robust that dogs display exaggerated gregariousness, referred to as hyper-sociability, which is a heightened propensity to initiate social contact that is often extended to members of another species, when compared with wolves into adulthood. Hyper-sociability, one facet of the domestication syndrome (5), is a multifaceted phenotype that may include extended proximity seeking and gaze (6, 7), heightened oxytocin levels (6), and inhibition of independent problem solving behavior in the presence of humans (8). This behavior is likely driven by behavioral neoteny, which is the extension of juvenile behaviors into adulthood and increases the ability for dogs to form primary attachments to social companions (4).

Due to strict selective breeding rules, distinct dog breeds conform to a predictable phenotype. It is this population structure and isolation that presents the dog as a powerful model for exploring the genetic underpinnings of complex traits such as behavior (9). Many dog breeds have been collectively scored using standardized tests for behavioral personality traits central to their domesticated nature (e.g., playfulness, sociability, aggression, trainability, curiosity or boldness) and breed-specific function (e.g., herding, pointing, chasing, working) (9, 10-17). Though there has been strong selection for breed conformation, inter-individual variation contribution to heritability estimates suggests that genetics plays a detectable role in shaping canine social behavior (18).

Phenotype evolution in the dog genome during the divergence process of dogs from wolves during domestication has been investigated through a genome-wide association scan of over 48,000 SNP genotypes from 701 dogs from 85 breeds, and 92 gray wolves with a Holarctic distribution (19). Using divergence, the top ranking outlier site was located within SLC24A4, a gene known to contain polymorphisms linked to eye and hair color variation in humans (19). The second ranking site was located within WBSCR17, a gene implicated in Williams-Beuren Syndrome (WBS) in humans. WBS is a neurodevelopmental disorder caused by a 1.5-1.8 Mb hemizygous deletion on human chromosome 7q11.23 spanning approximately 28 genes (20). This syndrome is characterized by delayed development, cognitive impairment, behavioral abnormalities, and hyper-sociability (21-23). A number of other studies have taken a different approach and targeted genes linked to social behavior in other taxa. For example, targeted variation was surveyed in the dopamine receptor D4 and tyrosine hydroxylase, both genes extensively studied for their roles in the primate brain's reward system (24). The study found an association between longer repeat polymorphisms with lowered activity and impulsivity in a limited survey of breeds. In a similar approach, variation surveyed at a regulatory SNP in the oxytocin receptor gene, also known to influence human pair bonding, was found to be associated with proximity seeking and friendliness in two dog breeds (25). However, behavioral genetic studies are still plagued with the challenge to understand the genetic architecture of nearly every facet of a complex behavior.

### SUMMARY

Disclosed herein are methods of identifying dogs or wolves with predispositions for hyper-social behavior, e.g., human-directed hyper-social behavior. The methods involve identifying structural variants at specific genetic loci within the Williams-Beuren Syndrome (WBS) locus on chromosome 6 of the dogs or wolves. In some embodiments, the structural variants include at least one of Cfa6.6, Cfa6.7, Cfa6.66, or Cfa6.83. In some embodiments, the structural variants include at least one of the genes GTF2I, GTF2IRD1, and WBSCR17.

Accordingly, disclosed herein is a method for predicting the probability of a dog or wolf exhibiting a sociable behavior comprising:
(a) genotyping a biological sample from a dog or wolf;
(b) counting the number of structural variants within the Williams-Beuren Syndrome (WBS) locus on canine chromosome 6; and
(c) predicting the probability of the dog or wolf exhibiting a sociable behavior based on the number of structural variants.

The disclosure herein allows for improved methods of ranking dogs or wolves according to their sociability. Thus, disclosed herein is a method of ranking dogs or wolves according to their likelihood of exhibiting a sociable behavior comprising:
(a) obtaining a biological sample from a first dog or wolf;
(b) determining the number of structural variants within the Williams-Beuren Syndrome (WBS) locus on chromosome 6 of the first dog or wolf;
(c) obtaining a biological sample from a second dog or wolf;
(d) determining the number of structural variants within the Williams-Beuren Syndrome (WBS) locus on chromosome 6 of the second dog or wolf; and
(e) ranking the first dog as being more likely to exhibit a sociable behavior than the second dog if the number of structural variants determined in step (b) is greater than the number of structural variants determined in step (d); or
(f) ranking the second dog as being more likely to exhibit a sociable behavior than the first dog if the number of structural variants determined in step (d) is greater than the number of structural variants determined in step (b).

In some embodiments, the biological sample is blood, saliva, cerebrospinal fluid, skin, or urine.

In some embodiments, genotyping the biological sample includes PCR amplification and agarose gel electrophoresis. In some embodiments, the genotyping utilizes at least one primer selected from the group consisting of:

(SEQ ID NO: 1)
CCCCTTCAGCCAGCATATAA,
(SEQ ID NO: 2)
TTCTCTGGGCTGTCTGGACT,
(SEQ ID NO: 3)
AAGTTTCTCTGATGGAAAACACA,
(SEQ ID NO: 4)
GGTGGCTGGAAATTTCAGTAG,
(SEQ ID NO: 5)
TGGAGCCATGATTAGGAAGG,
(SEQ ID NO: 6)
TAAGGAAGGACCCCATTTCC,
(SEQ ID NO: 7)
TGCTGCTTCATGTTCTGTGA,
(SEQ ID NO: 8)
TGGTGCATTAGCTTTGGTTG,
(SEQ ID NO: 9)
AACCACAGGAACAAAACCTCA,
and
(SEQ ID NO: 10)
CCTCCTGTTGGACATTTGGA.

In some embodiments, the structural variant is a transposable element that interrupts a gene in the WBS locus. In some embodiments, the transposable element is a retrotransposon. In some embodiments, the retrotransposon is a short interspersed nuclear element (SINE) or a long interspersed nuclear element (LINE).

In some embodiments, the method identifies at least one structural variant that occurs within at least one gene selected from the group consisting of GTF2I, GTF2IRD1, and WBSCR17.

In some embodiments, the social behavior is selected from the group consisting of attentional bias to social stimuli (ABS), hyper-sociability (HYP), and social interest in strangers (SIS).

In some embodiments, the methods disclosed herein include counting structural variants found at Cfa6.6, Cfa6.7, Cfa6.66, and Cfa6.83.

Disclosed herein is a method of screening a dog or wolf library comprising:
(a) obtaining a genomic library from a dog or wolf that contains the Williams-Beuren Syndrome (WBS) locus on canine chromosome 6;
(b) determining the number of structural variants in the WBS locus.

In some embodiments, the location of the structural variants is also determined.

In some embodiments, step (b) comprises determining the number of structural variants in at least one of GTF2I, GTF2IRD1, and WBSCR17. In some embodiments, step (b) comprises determining the number of structural variants in all of GTF2I, GTF2IRD1, and WBSCR17.

In some embodiments, step (b) comprises the use of the polymerase chain reaction (PCR) to amplify at least one DNA fragment from the WBS locus. In some embodiments, the DNA fragment comprises at least one of the loci Cfa6.6, Cfa6.7, Cfa6.66, or Cfa6.83.

In some embodiments, step (b) comprises the use of PCR to amplify the locus Cfa6.6 using the primers CCCCTTCAGCCAGCATATAA (SEQ ID NO: 1) (forward) and TTCTCTGGGCTGTCTGGACT (SEQ ID NO: 2) (reverse).

In some embodiments, step (b) comprises the use of PCR to amplify the locus Cfa6.6 using the primers AAGTTTCTCTGATGGAAAACACA (SEQ ID NO: 3) (forward) and GGTGGCTGGAAATTTCAGTAG (SEQ ID NO: 4) (reverse).

In some embodiments, step (b) comprises the use of PCR to amplify the locus Cfa6.7 using the primers TGGAGCCATGATTAGGAAGG (SEQ ID NO: 5) (forward) and TAAGGAAGGACCCCATTTCC (SEQ ID NO: 6) (reverse).

In some embodiments, step (b) comprises the use of PCR to amplify the locus Cfa6.66 using the primers TGCTGCTTCATGTTCTGTGA (SEQ ID NO: 7) (forward) and TGGTGCATTAGCTTTGGTTG (SEQ ID NO: 8) (reverse).

In some embodiments, step (b) comprises the use of PCR to amplify the locus Cfa6.83 using the primers AACCACAGGAACAAAACCTCA (SEQ ID NO: 9) (forward) and CCTCCTGTTGGACATTTGGA (SEQ ID NO: 10) (reverse).

In some embodiments, step (b) comprises the use of agarose gel electrophoresis to identify DNA fragments from the WBS locus that have altered mobility compared to the corresponding fragments from the dog reference genome and that are indicative of structural variants in the WBS locus from the library.

In some embodiments, step (b) comprises a hybridization step using at least one probe from the WBS locus that identifies structural variants in the WBS locus. In some embodiments, the hybridization step comprises fluorescence in-situ hybridization (FISH).

Also disclosed herein are canine breeding methods. The methods disclosed herein that allow for the prediction of sociability characteristics of canines permit breeders to select those canines for breeding that have desirable sociability characteristics. That is, by choosing canines for breeding that contain appropriate structural variants of the WBS locus, and by not choosing for breeding those canine that do not contain those variants, breeders can increase the likelihood that offspring will exhibit desirable sociability characteristics such as attentional bias to social stimuli (ABS), hyper-sociability (HYP), and social interest in strangers (SIS).

Over time, this can lead to the development of breeding lines of canines that are more suitable for certain roles; e.g., canines that are better family pets, because they are more attached to their owners. Similarly, undesirable traits such as aloofness or excessive aggression can be eliminated or reduced.

Accordingly, a further aspect of the disclosure herein is a method of producing dogs that are more likely to exhibit a sociable behavior comprising:
(a) selecting a male and female dog for breeding that each are known to have at least one structural variant within Cfa6.6, Cfa6.7, Cfa6.66, or Cfa6.83 in the Williams-Beuren Syndrome (WBS) locus; and
(b) mating the dogs of step (a) to produce offspring.

The disclosure herein also includes a method of producing dogs that are more likely to exhibit a sociable behavior comprising:
(a) genotyping male and female dogs for the presence of structural variants within the Williams-Beuren Syndrome (WBS) locus;
(b) selecting a male and female dog that each have at least one structural variant in Cfa6.6, Cfa6.7, Cfa6.66, or Cfa6.83 in the WBS locus; and
(c) mating the dogs of step (b) to produce offspring.

In some embodiments, the structural variant is at Cfa6.6, Cfa6.7, Cfa6.66, and Cfa6.83. In some embodiments, the structural variant occurs within at least one gene selected from the group consisting of GTF2I, GTF2IRD1, and WBSCR17.

Disclosed herein is a method of editing the genome of a dog comprising:
(a) obtaining a dog;
(b) using clustered regularly interspaced short palindromic repeats (CRISPRs)/CRISPR-associated (Cas) 9 to inactivate a gene in the Williams-Beuren Syndrome (WBS) locus on canine chromosome 6.

See Zou et al., Journal of Molecular Cell Biology (2015), 7(6), 580-58.

In some embodiments, the dog is obtained because it is desirable to increase the sociability of the dog.

In some embodiments, the gene is GTF2I, GTF2IRD1, or WBSCR17.

A further aspect of the disclosure herein is a kit for detecting the presence of structural variants within the Williams-Beuren Syndrome (WBS) locus of canines. The kit may comprise one or more primers suitable for use in PCR-based processes for detecting the structural variants. Such primers include:

(SEQ ID NO: 1)
CCCCTTCAGCCAGCATATAA,
(SEQ ID NO: 2)
TTCTCTGGGCTGTCTGGACT,
(SEQ ID NO: 3)
AAGTTTCTCTGATGGAAAACACA,
(SEQ ID NO: 4)
GGTGGCTGGAAATTTCAGTAG,
(SEQ ID NO: 5)
TGGAGCCATGATTAGGAAGG,
(SEQ ID NO: 6)
TAAGGAAGGACCCCATTTCC,
(SEQ ID NO: 7)
TGCTGCTTCATGTTCTGTGA,
(SEQ ID NO: 8)
TGGTGCATTAGCTTTGGTTG,
(SEQ ID NO: 9)
AACCACAGGAACAAAACCTCA,
and
(SEQ ID NO: 10)
CCTCCTGTTGGACATTTGGA.

In some embodiments, the kit comprises the primers CCCCTTCAGCCAGCATATAA (SEQ ID NO: 1) and TTCTCTGGGCTGTCTGGACT (SEQ ID NO: 2).

In some embodiments, the kit comprises the primers AAGTTTCTCTGATGGAAAACACA (SEQ ID NO: 3) and GGTGGCTGGAAATTTCAGTAG (SEQ ID NO: 4).

In some embodiments, the kit comprises the primers TGGAGCCATGATTAGGAAGG (SEQ ID NO: 5) and TAAGGAAGGACCCCATTTCC (SEQ ID NO: 6).

In some embodiments, the kit comprises the primers TGCTGCTTCATGTTCTGTGA (SEQ ID NO: 7) and TGGTGCATTAGCTTTGGTTG (SEQ ID NO: 8).

In some embodiments, the kit comprises the primers AACCACAGGAACAAAACCTCA (SEQ ID NO: 9) and CCTCCTGTTGGACATTTGGA (SEQ ID NO: 10).

In some embodiments, the kit further comprises instructions for use. In another embodiment, the primers are labeled using a detectable marker. The kit may further comprise at least one additional reagent such as buffers, dNTPs, DNA polymerases, DNA ligases, and restriction enzymes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Association of structural variants with indices of human-directed social behavior. A) Association with ABS, B) association with HYP, and C) association with SIS. Manhattan plots show statistical significance of each variant as a function of position in target region. Blue horizontal line denotes statistical significance to Bonferroni corrected level (p=2.38×10⁻³). Genic variants are green; intergenic variants are red.

FIG. 2. Association of structural variants with human-directed social behavior in multivariate regressions. A) Association in Behavioral Index model and B) Association in PC model. Manhattan plots show statistical significance of each variant as a function of position in target region. Blue horizontal line denotes significance to Bonferroni corrected level (p=2.38×10⁻³); dashed purple line denotes suggestive significance (p=0.01). Genic variants are green; intergenic variants are red.

FIG. 3. Differences between dogs and wolves for three behavioral indices used to predict the WBS phenotype. Stars indicate pairwise significant differences (p<0.05).

FIG. 4. Scree plot of principal components of human-directed social behavior. Plot shows variance in original data set (Table 16) explained by each PC.

FIG. 5. Scan for positive selection using a bivariate percentile score (XP-EHH and FST) to identify outliers (dashed line; bivariate score>2) indicated as sites in the 97.5th percentile. Annotated genes are indicated above the plot as black and gray bars, labeled with gene names.

FIG. 6. Gel electrophoresis banding patterns for three hyper-sociability-associated SV genotypes.

FIG. 7. A dot plot to represent the A) total number of insertions per population of species, and for each outlier locus B) Cfa6.6, C) Cfa6.7, D) Cfa6.66, and E) Cfa6.83. Underlined breeds have the “seeks attention” behavioral stereotype. (Abbreviations: Bernese Mountain dog, BMD; Border collie, BORD; Boxer, BOX; Basenji, BSNJ; Cairn terrier, CAIRN; WBS study dogs, Dog; Golden retriever, GOLD; Great Pyrenees, GPYR Jack Russell terrier, JACK; Alaska, malamute, MALA; Miniature poodle, MPOO; Miniature schnauzer, MSCHN; New Guinea singing dog, NGSD; Pariah dog, PARIAH, Saluki, SALU; Village dog, Village; Village dogs from Puerto Rico, Village_PR; Middle East, ME; North America, NA).

FIG. 8. Plots from the ANOVA of the total number of SV insertions at four outlier loci depend upon the population membership for A) residuals vs. fitted, B) Q-Q plot, C) scale location, and D) residuals vs. leverage.

FIG. 9. PCA from 25,510 unlinked genome-wide SNPs from the Affymetrix K9HDSNP array for six wolves and five dogs.

FIG. 10. SV Discovery Pipeline. Numbers represent steps within the pipeline as follows: 1) Deplexing and quality control, 2) Alignment to reference, 3) Variant calling, 4) SoftSearch SV discovery, 5) SVMerge SV discovery, 6) inGAP-SV SV discovery, 7) Filtering of SV, and 8) Merging of filtered SVs.

FIG. 11. Overlap in number of SVs identified by SVMerge, SoftSearch and inGAP-sv.

### DETAILED DESCRIPTION

“Detectable marker” refers to a moiety attached to an entity (such as a probe) to render the entity detectable. The moiety itself need not be detectable; it may become detectable upon reaction with yet another moiety. Detectable markers include fluorophores, chromophores, radioactive isotopes, chemiluminescent agents, haptens, and magnetic particles.

“Genotyping” refers to structural analysis of the Williams-Beuren Syndrome locus on canine chromosome 6 that provides information regarding the presence of structural variants in the WBS locus. Genotyping may be accomplished by any means known in the art, e.g., DNA sequencing, the use of PCR followed by agarose gel electrophoresis, or hybridization assays,

“Hyper-sociability” refers to a heightened propensity to initiate social contact that is often extended to members of another species.

The present inventors have determined that structural variants in genes associated with human Williams-Beuren Syndrome underlie stereotypical hyper-sociability in domestic dogs. Accordingly, disclosed herein are genetic variants associated with human-directed hyper-social behavior in domestic dogs and a method to detect the same.

A candidate locus associated with WBS in humans and known to be under positive selection in the domestic dog genome (19) was identified and resequenced. It was found that this region also harbors a large number of highly polymorphic structural variants (SVs) in canines, some of which are private to an individual dog or breed. This finding is concordant with the genetic heterogeneity of WBS in humans, where deletions range from 100 Kb to 1.8 Mb in size with variable breakpoints, attributed to chromosomal instability (42-44). SVs found in multiple individuals were identified that were significantly associated with one or more quantified behavioral traits informative on hyper-sociability and cognition.

Domestic dogs exhibit some of the key behavioral traits quantified in individuals with WBS, most notably hyper-sociability in the absence of superior social cognition. A 5 Mb genomic region on chromosome 6 previously found to be under positive selection in domestic dog breeds was analyzed by the present inventors. Deletion of this region in humans is linked to Williams-Beuren syndrome (WBS), a multi-system congenital disorder characterized by hyper-social behavior. Quantitative data on behavioral phenotypes symptomatic of WBS in humans were associated with structural changes in the WBS locus in dogs. It was found that hyper-sociability, a central feature of WBS, is also a core element of domestication that distinguishes dogs from wolves. Evidence is provided herein that structural variants in GTF2I and GTF2IRD1, genes previously implicated in the behavioral phenotype of patients with WBS and contained within the WBS locus, contribute to extreme sociability in dogs. This finding suggests that there are commonalities in the genetic architecture of WBS and canine tameness, and that directional selection may have targeted a unique set of linked behavioral genes of large phenotypic effect, allowing for rapid behavioral divergence of dog and wolf, facilitating co-existence with humans.

A third described gene, WBSCR17, has not been previously associated with sociability. However, this gene is up-regulated in cells treated with N-acetylglucosamine, a glucose derivative, suggesting a role in carbohydrate metabolism (54). SVs in WBSCR17 may represent an adaptation to a starch-rich diet typical of living in human settlements, a speculation concordant with a previous study (55).

Two of the SVs most associated with hyper-sociability, a trait uniquely displayed in domestic dogs among the canids, were SINE and LINE transposable elements, sub-types of retrotransposons that have high rates of insertion (e.g., 1 in 108 human births have a de novo L1 insertion; 56). With large phenotypic consequences due to the amplification of a few loci, these mobile elements have been implicated in the evolution of the canid genome (e.g., 57,58), as well as canine disease, syndromes, and morphology (e.g., 59-64).

These TEs were surveyed in an extended sampling of wild and domestic canines and found to be extremely rare in coyotes, while other insertions were derived and found only to segregate within domestic dogs. With a larger sample size and leveraging behavioral phenotypes from breed stereotypes, a significant association was found between TE copy number and behavior. Hence, it is conceivable that selection acting on hyper-sociability-associated TEs may have helped shape the evolution of the canid family. Canine WBS-linked SVs likely contribute to the developmental delay that facilitates ease of forming inter-species bonds and the juvenile-like hyper-sociability exhibited towards these social companions into adulthood. This coupling presents an intriguing parallel to the same processes observed in WBS affected individuals (20).

The genetic variants disclosed herein are associated with hyper-social behavior in domestic dogs and wolves, and will allow for a test to identify domestic dogs with predispositions for behavioral disorders or traits that make them more or less suited for placement in certain homes or working roles. This test might similarly be used in captive wolves to inform breeding practices. The disclosed approach allows for a commercial test to genotype dogs for the presence (or absence) of these genetic variants. In some embodiments, the disclosed test is a PCR-based test of specific genetic loci that are informative regarding the genetic influence for behavior.

A commercial genetic test employing the disclosed approach can genotype and count the number of genetic variants carried by each individual dog. The presence or absence of each variant can be assessed for a probability of how much more (or less) social the dog is as a direct result of the genotype, referred to as the allelic effect.

Some embodiments of the methods disclosed herein utilize primers or probes. Primers and probes may be oligonucleotides of at least 15 nucleotides in length. Primers are usually 15 base pairs to 100 base pairs in length, and preferably are 17 base pairs to 30 base pairs in length. The primer is not particularly limited as long as it is capable of amplifying at least a part of a DNA comprising the canine Williams-Beuren Syndrome locus on chromosome 6. The length of DNA which primers amplify is usually 15-1000 base pairs, preferably 20-500 base pairs, and more preferably 20-200 base pairs. When the oligonucleotide is used as a probe, its length is usually 5 base pairs to 200 base pairs, preferably 7 base pairs to 100 base pairs, more preferably 7 base pairs to 50 base pairs. The probe is not particularly limited as long as it is capable of hybridizing to a DNA comprising the canine Williams-Beuren Syndrome locus on chromosome 6.

In preferred embodiments, the primers are used in pairs that together amplify a region of the canine Williams-Beuren Syndrome locus on chromosome 6 that includes a structural variant. In preferred embodiments, the region is a region from at least one of GTF2I, GTF2IRD1, and WBSCR17.

In some embodiments, the probes hybridize to the canine Williams-Beuren Syndrome locus on chromosome 6 in which at least one of GTF2I, GTF2IRD1, and WBSCR17 does not contain a structural variant but do not hybridize to the canine Williams-Beuren Syndrome locus on chromosome 6 in which at least one of GTF2I, GTF2IRD1, and WBSCR17 does contain a structural variant. In some embodiments, the hybridization conditions are stringent hybridization conditions (see, for example, the conditions disclosed in Sambrook et al., Molecular Cloning, Cold Spring Harbor Laboratory Press, New York, USA, the 2nd edition, 1989).

A person of ordinary skill in the art would be able to design appropriate primers and probes for the methods disclosed herein based on the teachings herein with respect to GTF2I, GTF2IRD1, and WBSCR17 and the dog reference genome.

In some embodiments, the probes are immobilized on a solid phase. Examples of solid phases include, but are not limited to, microplate wells, plastic beads, nylon membranes, and magnetic particles.

### EXAMPLES

### Example 1—Solvable Tasks and Sociability Measures

The human-directed sociability of 18 domestic dogs and ten captive human-socialized gray wolves was evaluated using standard sociability (26,27) and problem solving tasks (2,8,28) commonly used to assess human-directed sociability in canines. Three sociability metrics were constructed to assess behaviors indicative of WBS (22): attentional bias to social stimuli (ABS), hyper-sociability (HYP), and social interest in strangers (SIS) (Tables 1, 2).

TABLE 1
Raw behavioral data.

| | ST-% | ST-% | ST-% | proximity | proximity | proximity | proximity |
|---|---|---|---|---|---|---|---|
| Animal | time look | time touch | time look | unfamiliar | unfamiliar | familiar | familiar |
| ID | box | box | human | passive (s) | active (s) | passive (s) | active (s) |
| 2768 | 15% | 5% | 4% | 24.72 | 87.96 | 64.08 | 105.6 |
| 2769 | 18% | 14% | 25% | 51.6 | 70.8 | 120 | 120 |
| 2770 | 8% | 6% | 17% | 30 | 120 | 114 | 112.8 |
| 2771 | 9% | 6% | 11% | 28.2 | 56.76 | 106.56 | 119.88 |
| 2772 | 4% | 3% | 1% | 85.2 | 112.8 | 99.6 | 117.6 |
| 2773 | 69% | 64% | 14% | 10.2 | 0 | 93.6 | 119.64 |
| 2774 | 100% | 97% | 0% | 3.12 | 4.8 | 69.72 | 103.2 |
| 2775 | 11% | 6% | 4% | 30 | 120 | 114 | 112.8 |
| 2776 | 4% | 3% | 33% | 43.44 | 117.96 | 109.32 | 119.64 |
| 2777 | 5% | 1% | 13% | 10.92 | 69.84 | 70.92 | 87.72 |
| 2778 | 5% | 4% | 12% | 27.6 | 120 | 76.8 | 120 |
| 2779 | 9% | 6% | 56% | 13.2 | 113.28 | 62.04 | 119.64 |
| 2780 | 10% | 4% | 32% | 21.48 | 113.64 | 71.52 | 117.72 |
| 2781 | 20% | 17% | 15% | 5.04 | 58.08 | 53.76 | 113.52 |
| 2782 | 25% | 16% | 34% | 14.16 | 65.04 | 68.88 | 119.88 |
| 2783 | 19% | 16% | 8% | 29.76 | 111.48 | 29.04 | 118.8 |
| 2784 | 18% | 12% | 7% | 61.2 | 119.88 | 120 | 119.28 |
| 2785 | 3% | 1% | 85% | 12 | 115.08 | 59.16 | 120 |

------------------------------------------------------------------------------------------------------------

| | | | | | | | |
|---|---|---|---|---|---|---|---|
| 2786 | 35% | 30% | 1% | 49.2 | 119.16 | 65.52 | 98.4 |
| 2787 | 100% | 100% | 0% | 18.36 | 65.4 | 32.76 | 106.08 |
| 2788 | 90% | 94% | 0% | 44.76 | 0 | 24.6 | 17.76 |
| 2789 | 97% | 98% | 0% | 36.36 | 0 | 21.72 | 53.4 |
| 2790 | 99% | 100% | 0% | 108.24 | 71.28 | 0 | 82.56 |
| 2791 | 83% | 81% | 0% | 60 | 104.52 | 1.8 | 0 |
| 2792 | 100% | 99% | 0% | 48.84 | 0 | 24.36 | 95.4 |
| 2793 | 100% | 98% | 0% | 17.64 | 7.56 | 0.24 | 114.96 |
| 2794 | 100% | 98% | 0% | — | — | — | — |
| 2795 | 100% | 90% | 0% | 45.96 | 113.64 | 69.48 | 119.4 |

Dashed line separates dogs (above) from wolves (below).

TABLE 2
Data for indices of human-directed social behavior.

| Animal ID | ABS | HYP | SIS | PC1 | PC2 | PC3 |
|---|---|---|---|---|---|---|
| 2769 | 0.864 | 362.40 | 122.4 | −1.321 | 0.453 | −0.764 |
| 2771 | 0.823 | 311.40 | 84.96 | −1.041 | −0.211 | −1.030 |
| 2772 | 0.326 | 415.20 | 198 | −0.944 | 2.244 | −0.936 |
| 2773 | 0.424 | 223.44 | 10.2 | 0.214 | −1.634 | −1.214 |
| 2774 | 0.000 | 180.84 | 7.92 | 1.289 | −1.681 | −1.083 |
| 2775 | 0.548 | 376.80 | 150 | −1.412 | 0.645 | −0.802 |
| 2776 | 1.246 | 390.36 | 161.4 | −1.973 | 0.638 | −0.001 |
| 2777 | 1.031 | 239.40 | 80.76 | −0.520 | −0.470 | 0.089 |
| 2778 | 0.995 | 344.40 | 147.6 | −1.320 | 0.367 | −0.106 |
| 2779 | 1.188 | 308.16 | 126.48 | −1.920 | −0.739 | 1.395 |
| 2780 | 1.067 | 324.36 | 135.12 | −1.518 | −0.156 | 0.563 |
| 2781 | 0.704 | 230.40 | 63.12 | −0.423 | −1.059 | −0.032 |
| 2782 | 0.863 | 267.96 | 79.2 | −0.983 | −0.987 | 0.278 |
| 2783 | 0.585 | 289.08 | 141.24 | −0.416 | 0.239 | 0.397 |
| 2784 | 0.538 | 420.36 | 181.08 | −1.330 | 1.498 | −0.984 |
| 2785 | 1.393 | 306.24 | 127.08 | −2.545 | −1.124 | 2.356 |

-------------------------------------------------------------------------------------------

| | | | | | | |
|---|---|---|---|---|---|---|
| 2786 | 0.167 | 332.28 | 168.36 | −0.174 | 1.155 | −0.108 |
| 2787 | 0.000 | 222.60 | 83.76 | 1.250 | −0.689 | −0.187 |
| 2788 | 0.000 | 87.12 | 44.76 | 3.086 | 0.020 | 0.532 |
| 2789 | 0.000 | 111.48 | 36.36 | 2.687 | −0.496 | 0.131 |
| 2790 | 0.000 | 262.08 | 179.52 | 2.404 | 2.168 | 0.415 |
| 2791 | 0.000 | 166.32 | 164.52 | 2.690 | 1.662 | 1.815 |
| 2792 | 0.000 | 168.60 | 48.84 | 2.224 | −0.400 | −0.474 |
| 2793 | 0.000 | 140.40 | 25.2 | 1.995 | −1.442 | −0.249 |

Dashed lines separates dogs (above) from wolves (below).

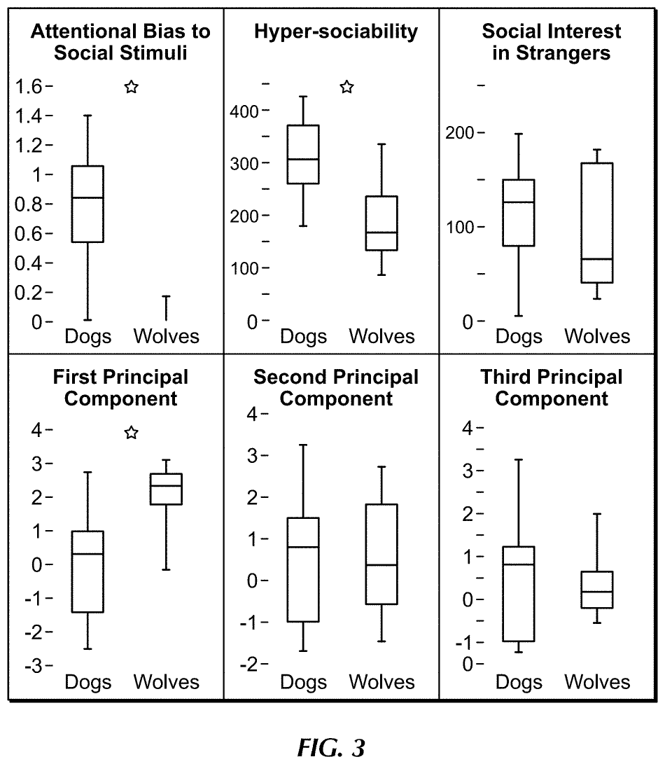

Solvable task performance was used to assess attentional bias towards social stimuli and independent problem solving performance (independent physical cognition). Subjects were given up to two minutes to open a solvable puzzle box (8) that contained half of a 2.5 cm thick piece of summer sausage, both when alone and with a neutral human present. The trial was considered complete after meeting one of the following conditions: the puzzle box lid was completely removed, the food obtained, or two minutes elapsed. All trials were video recorded and coded for whether the puzzle box was solved and the time to solve it. To compare attention towards the puzzle box versus social stimuli in the human-present condition, the percentage of time spent looking at the puzzle box, touching the puzzle box, and looking at the human was recorded (8). An independent researcher, who was blind to the purpose of this study, coded 30% of the videos, and found that inter-rater reliability was very strong (weighted Cohen's kappa, K 0.98; 95% confidence interval: 0.97-0.99). Domestic dogs spent a significantly greater proportion of trial time gazing at the human when compared to wolves when a human was present during the solvable task (median gaze towards human: dog=21%, wolf=0%; Two tailed Mann-Whitney, n_{dog}=18, n_{wolf}=10, U=6, p<0.0001). Dogs also spent a significantly smaller proportion of trial time looking at the puzzle box (median gaze towards box: dog=10%, wolf=100%; Two tailed Mann-Whitney, n_{dog}=18, n_{wolf}=10, U=171.5, p=0.0001) and a significantly smaller proportion of trial time trying to solve the puzzle (median dog=6%, wolf=98%; Two tailed Mann-Whitney, n_{dog}=18, n_{wolf}=10, U=175, p<0.0001) compared to wolves, a finding that has been equated with social inhibition of problem solving behavior in both the canine (9) and human WBS literature (22). Significantly more wolves successfully solved the task when compared to dogs in both the human present and alone conditions (Human present: 2/18 dogs successful, 8/10 wolves successful, Two-tailed Fisher's exact test, p=0.0005; Alone: 2/18 dogs successful, 9/10 wolves successful, Two-tailed Fisher's exact test, p=0.0001). Overall, concordant with WBS, dogs displayed greater ABS than wolves, corresponding to a reduction in independent problem solving success (FIG. 3).

The sociability test measured human-directed proximity seeking behavior, and was assessed by comparing total sociability scores across all sociability conditions. Each phase occurred twice, once with an unfamiliar human and once with a familiar human, totaling four phases run over eight consecutive minutes. In all phases, the experimenter sat on a familiar chair (dogs) or bucket (wolves) inside a marked circle of 1 m circumference denoting proximity. During the passive phase, the experimenter sat quietly on the chair or bucket and ignored the subject by looking down toward the floor. If the animal sought physical contact, then the experimenter touched the subject twice, but did not speak or make eye contact with the animal. During the active phase, the experimenters called the animals by name and actively encouraged contact while remaining in their designated location. Dogs spent more time in proximity to humans than did wolves (median percent of time spent within 1 m of humans: dogs=65%, wolves=35%; Two tailed Mann-Whitney, n_{dog}=18, n_{wolf}=9, U=30, p<0.005). Dog and wolf sociability towards an unfamiliar human was used to assess social interest in strangers. Dogs spent more time within 1 m of a stranger when compared to wolves (median dogs=53%, wolves=28%) however this difference was not statistically significant (Two tailed Mann-Whitney, n_{dog}=18, n_{wolf}=9, U=76, p=0.51). In summary, dogs were hyper-social compared to wolves, although there was no significant difference in their social interest in strangers (FIG. 3).

The dimensionality of six behavioral traits (Table 3) were reduced to three components that are orthogonal and uncorrelated to each other, whereas ABS, HYP and SIS are correlated.

TABLE 3
Loadings of first three principal components
of human-directed social behavior.

| Behavior | PC1 | PC2 | PC3 |
|---|---|---|---|
| Proportion of time look human | −0.386 | −0.269 | 0.631 |
| Proportion of time look object | 0.536 | −0.153 | −0.066 |
| Proximity unfamiliar passive | 0.153 | 0.782 | −0.061 |
| Proximity unfamiliar active | −0.400 | 0.490 | 0.339 |
| Proximity familiar passive | −0.444 | 0.084 | −0.554 |
| Proximity familiar active | −0.429 | −0.213 | −0.414 |

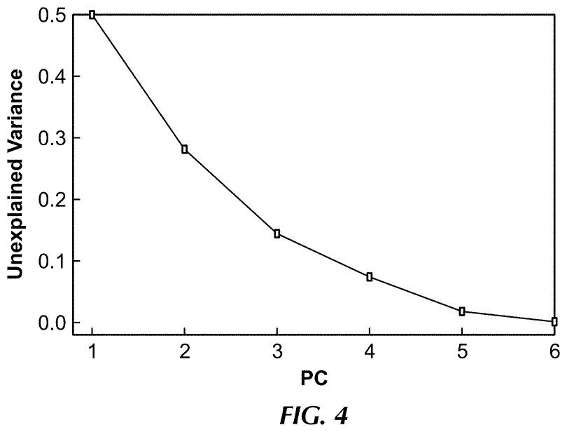

Principal Components 1, 2, and 3 accounted for 50%, 22%, and 14% of total behavioral variation, respectively. Both KMO (KMO=0.62, with values>0.6 recommended as informative) and Bartlett's test, which was significant (X2(15)=60.42, p=2.13×10⁻⁰⁷) were calculated. Analysis of the loadings of the constituent behaviors (Table 3; FIG. 3) indicated that PC1 represents an autonomous or independent phenotype, as this component is negatively correlated with all behaviors associated with human-directed sociability with the exception of proximity unfamiliar passive. PC1 also had positive loadings from time look object, a measure indicating a lack of attentional bias to social stimuli (FIG. 4). Loadings of each behavior were roughly equal, with the exception of proximity unfamiliar passive, which had a loading approximately one third the average magnitude of the others. PC2's loadings were heavily biased towards, and positively associated with, the measures of proximity to an unfamiliar person (average loading of 0.64, as compared to an average loading of −0.14 for the other loadings), suggesting that PC2 reflects boldness. The biological meaning of PC3 is more difficult to interpret, but given that it is strongly and positively loaded by the behavior time look human (loading of 0.63 compared to an average loading for all other factors of −0.15), it predominantly reflects reliance on humans in the solvable task test. As expected given interpretation of PC1 as socially inhibited phenotype, dogs had lower PC1 values than wolves (Mann-Whitney U-test: U=3, p<0.00005, median: dogs=−1.18, wolves=2.31). Dogs and wolves did not have significantly different values for PC2 (Mann-Whitney U-test: U=54, p=0.57, median: dogs=−0.18, wolves=−0.19) or for PC3 (Mann-Whitney U-test: U=48, p=0.35, median: dogs=−0.069 wolves=0.011).

### Example 2—De Novo Annotation of Structural Variants

In a subset of animals with quantitative behavioral data (n_{dog}=16; n_{wolf}=8), paired-end 2×67 nt sequence data were collected from 5 Mb spanning the candidate canine WBS locus on canine chromosome 6 (2,031,491-7,215,670 bp) which contains 46 annotated genes, 27 of which are in the human WBS locus (Tables 4, 5). The target region had an average of 15.5-fold sequence coverage (dogs: 15.2; wolves: 16.0) (Table 4).

TABLE 4
Sample information and the total number of raw reads compared to the number of
processed reads after using cutadapt to trim/clip paired end sequences. Average
sequence coverage is for target region chromosome 6 (2,031,491-7,215,670 bp).

| | | | | | | | | Mean | |
|---|---|---|---|---|---|---|---|---|---|
| | | | | | | No. of | Prop. of | library | |
| Sample | Species | | | | No. of | reads post- | reads | insert | Mean |
| ID | Membership | Breed | Age | Sex | raw reads | processing | dropped | size (bp) | coverage* |
| 2769 | Domestic | Mix | 4 | F | 20127088 | 20066328 | 0.003 | 304 | 15.4 |
| | dog | | | | | | | | |
| 2771 | Domestic | Mix | 2 | F | 18501656 | 18448868 | 0.003 | 271 | 14.5 |
| | dog | | | | | | | | |
| 2772 | Domestic | Russian | 5 | F | 17305060 | 17251544 | 0.003 | 304 | 12.6 |
| | dog | Terrier | | | | | | | |
| 2773 | Domestic | Dachshund | 6 | F | 21494600 | 21434496 | 0.003 | 281 | 17.7 |
| | dog | | | | | | | | |
| 2774 | Domestic | Weimaraner | 6 | M | 20697840 | 20634212 | 0.003 | 256 | 15.6 |
| | dog | | | | | | | | |
| 2775 | Domestic | Mix | 6 | M | 19885276 | 19821756 | 0.003 | 275 | 16.2 |
| | dog | | | | | | | | |
| 2776 | Domestic | Golden | 10 | F | 19910172 | 19847944 | 0.003 | 279 | 14.3 |
| | dog | Retriever | | | | | | | |
| 2777 | Domestic | Labrador | 3 | M | 30074496 | 29994680 | 0.003 | 259 | 17.2 |
| | dog | Retriever | | | | | | | |
| 2778 | Domestic | Mix | 2 | M | 24926916 | 24854488 | 0.003 | 274 | 18.5 |
| | dog | | | | | | | | |
| 2779 | Domestic | Mix | 1 | M | 19283016 | 19227644 | 0.003 | 282 | 13.8 |
| | dog | | | | | | | | |
| 2780 | Domestic | Mix | 11 | F | 18985824 | 18928344 | 0.003 | 272 | 12.2 |
| | dog | | | | | | | | |
| 2781 | Domestic | Mix | 6 | M | 22208112 | 22140900 | 0.003 | 269 | 18.4 |
| | dog | | | | | | | | |
| 2782 | Domestic | Saluki | 2 | M | 20127088 | 20066328 | 0.003 | 304 | 15.4 |
| | dog | | | | | | | | |
| 2783 | Domestic | Mix | 3 | M | 18501656 | 18448868 | 0.003 | 271 | 14.5 |
| | dog | | | | | | | | |
| 2784 | Domestic | Mix | 2 | M | 18294764 | 18238376 | 0.003 | 261 | 11.7 |
| | dog | | | | | | | | |
| 2785 | Domestic | Mix | 4 | F | 19086720 | 19034004 | 0.003 | 261 | 15.1 |
| | dog | | | | | | | | |
| 2786 | Gray wolf | NA | 8 | F | 19333428 | 19275892 | 0.003 | 264 | 13.9 |
| 2787 | Gray wolf | NA | 3 | M | 21307104 | 21243032 | 0.003 | 262 | 16.0 |
| 2788 | Gray wolf | NA | 7 | M | 20928148 | 20866492 | 0.003 | 263 | 13.8 |
| 2789 | Gray wolf | NA | 3 | F | 22880760 | 22818112 | 0.003 | 270 | 17.5 |
| 2790 | Gray wolf | NA | 14 | F | 19837444 | 19779788 | 0.003 | 264 | 16.6 |
| 2791 | Gray wolf | NA | 8 | F | 20472512 | 20415648 | 0.003 | 276 | 14.5 |
| 2792 | Gray wolf | NA | 2 | F | 23722756 | 23652336 | 0.003 | 267 | 18.2 |
| 2793 | Gray wolf | NA | 3 | M | 21855032 | 21776192 | 0.004 | 258 | 17.3 |

(Abbreviations: female, F; North America, NA; male, M)
*After PCR duplications were removed.

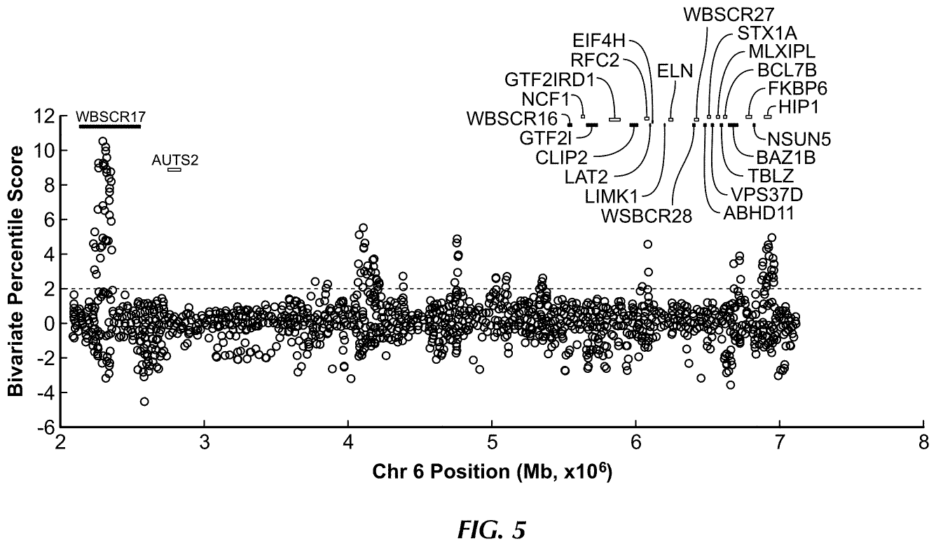

Genotypes for 26,296 SNPs were obtained, which were further filtered to retain 4,844 SNPs with non-missing polymorphic data (average density of 1 SNP every 14.4 Kb). To confirm this region as containing species-specific variation, it was determine if this region displays signals of positive selection in the dog genome, an effort to independently validate the original finding (19). The composite bivariate percentile score was calculated and confirmed that the candidate gene, WBS Chromosome Region 17 (WBSCR17), is under positive selection as a domestication candidate and was significantly depleted of heterozygosity in the dog (mean H_{O}: dog=0.01, wolf=0.37; 1-tailed t-test with unequal variance, p=7.4×10⁻³⁸) (FIG. 5; Table 5).

TABLE 5
Outlier clusters on chromosome 6 (canfam3.1) showing signals of positive selection from XP-EHH.

| | | | | No. | | Signal of | |
|---|---|---|---|---|---|---|---|
| Cluster | | | Median | outlier | Average H_{O} in dog/wolf | selection in | |
| ID | Start | Stop | BVS | SNPs | (t test p-value) | genome of: | Genes |
| 6.1 | 2,226,371 | 2,352,136 | 7.34 | 54 | 0.01/0.37 (7.4 × 10⁻³⁸) | Dog | WBSCR17 |
| 6.2 | 3,769,529 | 3,858,530 | 2.27 | 3 | 0.40/0.00 (1.4 × 10⁻³) | Wolf | |
| 6.3 | 4,064,791 | 4,215,649 | 2.89 | 36 | 0.24/0.00 (5.8 × 10⁻⁶) | Wolf | |
| 6.4 | 4,739,462 | 4,766,313 | 3.96 | 7 | 0.11/0.25 (1.3 × 10⁻²) | Dog | |
| 6.5 | 5,023,335 | 5,102,019 | 2.58 | 8 | 0.04/0.47 (3.4 × 10⁻⁴) | Dog | |
| 6.6 | 5,341,689 | 5,351,682 | 2.17 | 8 | 0.03/0.59 (7.9 × 10⁻⁶) | Dog | |
| 6.7 | 5,351,682 | 6,085,558 | 2.96 | 3 | 0.25/0.0 (0.012) | Wolf | CLIP2 |
| 6.8 | 6,679,302 | 6,728,731 | 3.62 | 5 | 0.00/0.38 (7.4 × 10⁻⁸) | Dog | BAZ1B |
| 6.9 | 6,866,332 | 6,955,315 | 2.74 | 35 | 0.43/0.10 (7.4 × 10⁻⁸) | Wolf | FKBP6, |
| | | | | | | | NSUN5 |

Abbreviations: bivariate percentile score, BPS; observed heterozygosity, Ho).
P-values from a 1-tailed t-test of unequal variance are provided in parentheses.

As this candidate region shows structural variation (SV) linked to WBS in humans (20), and is known to vary widely in its functional consequences (e.g., neurodevelopmental diseases [29]; autism spectrum disorders [30]), in silico SV annotation in the dog and wolf genomes was completed using three programs—SVMerge (31), SoftSearch (32), and inGAP-SV (33), which together utilize all available SV detection algorithms: read pair (RP), short reads (SR), read depth (RD), and assembly-based (AS). 38 deletions, 30 insertions, 13 duplications, six transpositions, a single inversion, and one complex variant relative to the reference dog genome were annotated (Tables 6, 7).

TABLE 6
Summary of de novo annotated structural
variants on canine chromosome 6.

| | | Substituent SV Detection Programs: | | | |
|---|---|---|---|---|---|
| | | SVMerge | SoftSearch | InGAP-sv | Total |
| Number of | Raw | 120 | 126 | 112 | 358 |
| Structural | Post- | 96 | 111 | 70 | 277 |
| Variations | Filtering | | | | |
| | Merged | | | | 89 |

TABLE 7
De novo annotated structural variants on canine chromosome
6 (coordinates based on canfam3.1 assembly).

| Locus | | | | | | |
|---|---|---|---|---|---|---|
| ID | Type | Start | Size (bp) | f(Dogs) | f(Wolves) | Gene |
| Cfa6.1 | DEL | 2,095,386 | 638,909 | 0.06 | 0.00 | WBSCR17, |
| | | | | | | GLNT9, |
| | | | | | | AUTS2 |
| Cfa6.2 | INS | 2,140,817 | 341 | 0.00 | 0.13 | WBSCR17 |
| Cfa6.3 | INS | 2,141,493 | 76 | 0.25 | 0.38 | WBSCR17 |
| Cfa6.4 | INS | 2,205,140 | 11 | 0.06 | 0.00 | WBSCR17 |
| Cfa6.5 | DEL | 2,432,140 | 1,800,342 | 0.06 | 0.00 | WBSCR17, |
| | | | | | | AUTS2 |
| Cfa6.6 | DEL | 2,521,650 | 198 | 0.00 | 0.88 | WBSCR17 |
| Cfa6.7 | DEL | 2,546,359 | 235 | 0.06 | 0.38 | WBSCR17 |
| Cfa6.8 | INS | 2,583,455 | 58 | 0.00 | 0.88 | |
| Cfa6.9 | DEL | 2,625,969 | 218 | 0.06 | 0.63 | |
| Cfa6.10 | INS | 2,689,902 | 7 | 0.00 | 0.13 | |
| Cfa6.11 | DUP | 2,734,984 | 2,347,334 | 0.00 | 0.13 | AUTS2 |
| Cfa6.12 | DEL | 3,009,985 | 627,053 | 0.00 | 0.13 | AUTS2 |
| Cfa6.13 | DUP | 3,010,060 | 626,977 | 0.00 | 0.13 | AUTS2 |
| Cfa6.14 | DUP | 3,010,100 | 2,121,751 | 0.00 | 0.13 | AUTS2 |
| Cfa6.15 | DUP | 3,012,589 | 2,239,167 | 0.00 | 0.13 | AUTS2 |
| Cfa6.16 | DEL | 3,018,553 | 687 | 0.06 | 0.00 | AUTS2 |
| Cfa6.17 | DEL | 3,209,048 | 1,279 | 0.25 | 0.00 | AUTS2 |
| Cfa6.18 | DEL | 3,241,300 | 694 | 0.06 | 0.00 | AUTS2 |
| Cfa6.19 | DEL | 3,452,567 | 715 | 0.06 | 0.00 | AUTS2 |
| Cfa6.20 | DEL | 3,452,997 | 294 | 0.31 | 0.25 | AUTS2 |
| Cfa6.21 | INS | 3,589,775 | 0 | 0.06 | 0.00 | AUTS2 |
| Cfa6.22 | DUP | 3,633,739 | 1,338,749 | 0.25 | 0.13 | AUTS2 |
| Cfa6.23 | INS | 3,875,594 | 351 | 0.06 | 0.00 | AUTS2 |
| Cfa6.24 | DEL | 3,976,412 | 223 | 0.31 | 0.00 | |
| Cfa6.25 | TRA | 3,986,929 | 513 | 0.56 | 0.88 | |
| Cfa6.26 | DEL | 3,986,940 | 536 | 0.88 | 0.63 | |
| Cfa6.27 | INS | 4,194,480 | 22 | 0.00 | 0.38 | |
| Cfa6.28 | INS | 4,231,965 | 3 | 0.31 | 0.13 | |
| Cfa6.29 | DEL | 4,232,120 | 351 | 0.50 | 0.38 | |
| Cfa6.30 | INS | 4,272,655 | 13 | 0.13 | 0.00 | |
| Cfa6.31 | DUP | 4,312,149 | 638 | 0.13 | 0.00 | |
| Cfa6.32 | INS | 4,358,450 | 368 | 0.81 | 0.75 | |
| Cfa6.33 | DUP | 4,369,908 | 888 | 0.13 | 0.00 | |
| Cfa6.34 | DEL | 4,370,055 | 448 | 0.00 | 0.13 | |
| Cfa6.35 | DEL | 4,370,264 | 246 | 0.25 | 0.63 | |
| Cfa6.36 | DUP | 4,377,190 | 859 | 0.13 | 0.00 | |
| Cfa6.37 | DEL | 4,486,820 | 470 | 0.00 | 0.13 | |
| Cfa6.38 | DUP | 4,514,366 | 1,067 | 0.13 | 0.00 | |
| Cfa6.39 | DEL | 4,514,621 | 551 | 0.06 | 0.13 | |
| Cfa6.40 | TRA | 4,514,643 | 573 | 0.31 | 0.50 | |
| Cfa6.41 | DEL | 4,691,721 | 216 | 0.00 | 0.25 | |
| Cfa6.42 | INS | 4,766,960 | 73 | 0.13 | 0.50 | |
| Cfa6.43 | INS | 4,767,241 | 65 | 0.06 | 0.00 | |
| Cfa6.44 | INS | 4,767,367 | 363 | 0.88 | 1.00 | |
| Cfa6.45 | DUP | 4,792,086 | 646 | 0.13 | 0.00 | |
| Cfa6.46 | INV | 4,839,392 | 3,328 | 0.00 | 0.13 | |
| Cfa6.47 | DEL | 4,842,442 | 225 | 0.25 | 0.88 | |
| Cfa6.48 | DUP | 4,858,013 | 622 | 0.13 | 0.00 | |
| Cfa6.49 | DUP | 4,910,429 | 578 | 0.13 | 0.00 | |
| Cfa6.50 | INS | 5,042,932 | 366 | 0.38 | 0.88 | |
| Cfa6.51 | DEL | 5,065,830 | 14,796 | 0.06 | 0.00 | |
| Cfa6.52 | DEL | 5,089,612 | 213 | 0.00 | 0.13 | |
| Cfa6.53 | DUP | 5,213,911 | 810 | 0.13 | 0.00 | |
| Cfa6.54 | DEL | 5,214,176 | 381 | 0.44 | 0.63 | |
| Cfa6.55 | DEL | 5,277,159 | 2,053 | 0.00 | 0.25 | |
| Cfa6.56 | INS | 5,318,593 | 30 | 0.31 | 0.50 | |
| Cfa6.57 | DEL | 5,337,423 | 226 | 0.00 | 0.75 | |
| Cfa6.58 | INS | 5,346,453 | 17 | 0.00 | 0.13 | |
| Cfa6.59 | INS | 5,634,780 | 456 | 0.94 | 0.88 | CBX3 |
| Cfa6.60 | D_I | 5,646,194 | 118 | 0.44 | 0.38 | |
| Cfa6.61 | INS | 5,646,321 | 294 | 0.13 | 0.00 | |
| Cfa6.62 | INS | 5,646,326 | 21 | 0.06 | 0.00 | |
| Cfa6.63 | INS | 5,646,624 | 3 | 0.00 | 0.13 | |
| Cfa6.64 | INS | 5,652,383 | 4 | 0.06 | 0.13 | |
| Cfa6.65 | TRA | 5,682,203 | 226 | 0.00 | 0.13 | GTF2IRD2 |
| Cfa6.66 | DEL | 5,753,703 | 290 | 0.69 | 0.13 | GTF2I |
| Cfa6.67 | TRA | 5,753,734 | 265 | 0.13 | 0.00 | GTF2I |
| Cfa6.68 | DEL | 5,820,166 | 231 | 0.38 | 0.63 | GTF2I |
| Cfa6.69 | INS | 5,844,759 | 49 | 0.31 | 0.50 | |
| Cfa6.70 | INS | 5,845,117 | 3 | 0.00 | 0.13 | |
| Cfa6.71 | INS | 5,859,196 | 3 | 0.06 | 0.00 | |
| Cfa6.72 | DEL | 5,902,332 | 715 | 0.19 | 0.13 | GTF2IRD1 |
| Cfa6.73 | DEL | 6,016,951 | 254 | 0.06 | 0.00 | |
| Cfa6.74 | TRA | 6,016,969 | 207 | 0.06 | 0.00 | |
| Cfa6.75 | INS | 6,289,609 | 345 | 0.13 | 0.25 | |
| Cfa6.76 | DEL | 6,399,238 | 266 | 0.00 | 0.13 | |
| Cfa6.77 | DEL | 6,400,822 | 216 | 0.31 | 0.75 | |
| Cfa6.78 | DEL | 6,522,289 | 234 | 0.25 | 0.38 | |
| Cfa6.79 | DEL | 6,718,167 | 263 | 0.25 | 0.38 | BAZIB |
| Cfa6.80 | DEL | 6,718,220 | 10,206 | 0.00 | 0.13 | BAZIB |
| Cfa6.81 | INS | 6,795,640 | 0 | 0.06 | 0.00 | |
| Cfa6.82 | INS | 6,889,833 | 10 | 0.00 | 0.38 | NSUN5 |
| Cfa6.83 | DEL | 6,914,106 | 222 | 0.19 | 0.50 | POM121 |
| Cfa6.84 | TRA | 6,947,879 | 260 | 0.06 | 0.00 | |
| Cfa6.85 | DEL | 6,947,889 | 247 | 0.00 | 0.13 | |
| Cfa6.86 | INS | 7,156,514 | 336 | 0.25 | 0.13 | |
| Cfa6.87 | DEL | 7,194,197 | 174 | 0.00 | 0.13 | |
| Cfa6.88 | DEL | 7,378,268 | 479 | 0.13 | 0.00 | STYXL1 |
| Cfa6.89 | INS | 7,378,904 | 2 | 0.06 | 0.00 | STYXL1 |

Abbreviations: D_I, deletion-insertion; DEL, deletion; DUP, duplication; INS, insertion; INV, inversion; TRA, translocation; SV, structural variant; f, frequency.

There was considerable private variation, with 31 annotated SVs found only in dogs, 26 found only in wolves, and a level of heterogeneity observed in wolves that is comparable to that found in human WBS (34) (mean n: wolf=21, dog=15, 2-tailed t-test p=0.026) (Table 8).

TABLE 8
Structural variant summary statistics per individual.

| | | | | % Target |
|---|---|---|---|---|
| | | | # Nucleotides | Region |
| | Species | | Affected | Affected |
| Sample ID | Membership | # SVs | by SVs | by SVs |
| 2769 | Domestic Dog | 20 | 9,853 | 0.19% |
| 2771 | Domestic Dog | 10 | 2,949 | 0.06% |
| 2772 | Domestic Dog | 9 | 1,339,332 | 25.83% |
| 2773 | Domestic Dog | 16 | 3,287 | 0.06% |
| 2774 | Domestic Dog | 18 | 641,634 | 12.38% |
| 2775 | Domestic Dog | 15 | 3,330 | 0.06% |
| 2776 | Domestic Dog | 13 | 2,939 | 0.06% |
| 2777 | Domestic Dog | 14 | 4,264 | 0.08% |
| 2778 | Domestic Dog | 16 | 1,339,843 | 25.84% |
| 2779 | Domestic Dog | 12 | 3128 | 0.06% |
| 2780 | Domestic Dog | 7 | 1,801,588 | 34.75% |
| 2781 | Domestic Dog | 22 | 1341938 | 25.89% |
| 2782 | Domestic Dog | 20 | 9,853 | 0.19% |
| 2783 | Domestic Dog | 9 | 2,709 | 0.05% |
| 2784 | Domestic Dog | 13 | 4,178 | 0.08% |
| 2785 | Domestic Dog | 18 | 1,356,209 | 26.16% |
| Average Across Dogs | | 15 | 491,690 | 9.48% |
| 2786 | Gray Wolf | 17 | 3,089 | 0.06% |
| 2787 | Gray Wolf | 19 | 633,662 | 12.22% |
| 2788 | Gray Wolf | 9 | 1,349,796 | 26.04% |
| 2789 | Gray Wolf | 31 | 6,642 | 0.13% |
| 2790 | Gray Wolf | 27 | 2,351,731 | 45.36% |
| 2791 | Gray Wolf | 20 | 2,240,258 | 43.21% |
| 2792 | Gray Wolf | 23 | 2,124,153 | 40.97% |
| 2793 | Gray Wolf | 25 | 6,491 | 0.13% |

| | | | |
|---|---|---|---|
| Average Across Wolves | 21 | 1,089,478 | 21.02% |
| Average Across all Animals | 17 | 690,952 | 13.33% |

### Example 3—Candidate Region Association Test

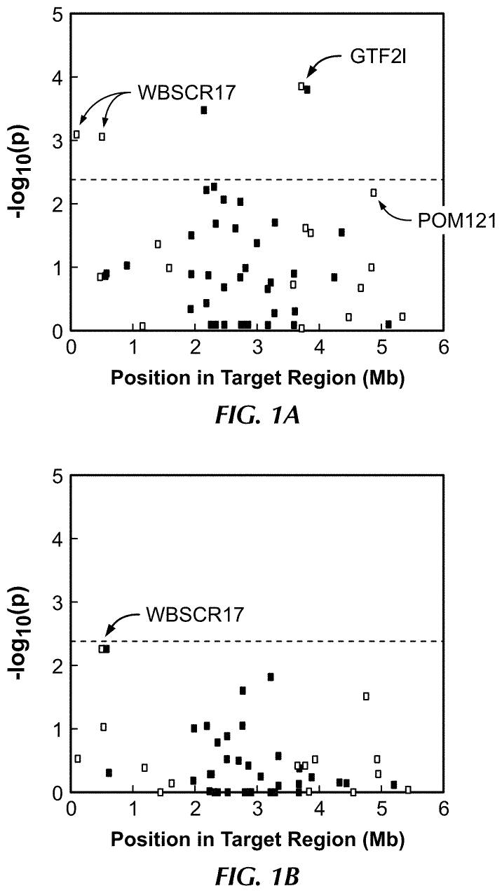

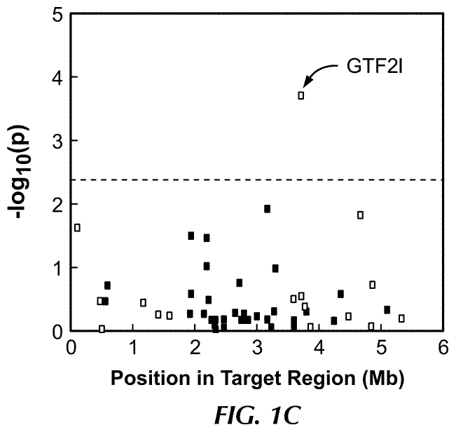

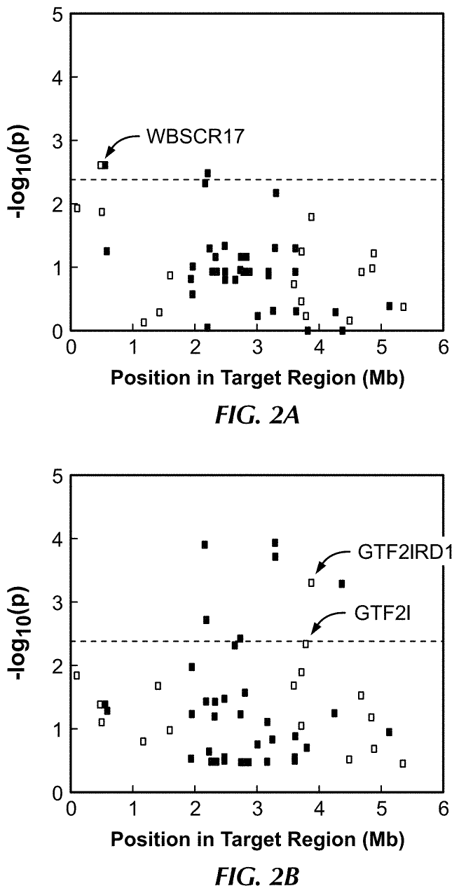

Linear mixed models were used to determine the association of SVs with human-directed sociability. Three univariate models were tested for their association with each of the three behavioral indices (ABS, HYP, SIS) (FIG. 1). In addition, association of SVs with the three behavioral indices collectively was tested for, referred to as the Behavioral index model, and separately with a model that included the first three principal components (PC model) describing human-directed sociable behavior (FIG. 2). Four genic SVs were significantly associated with human-directed social behavior (adjusted p<2.38×10⁻³): one SV within GTF2I (Cfa6.66); one SV within GTF2IRD1 (Cfa6.72); and two within WBSCR17 associated with ABS (Cfa6.3 and Cfa6.7) (Table 9).

TABLE 9
Genic loci associated with indices of human-directed social behavior across dogs and wolves.

| | | | | | % | | |
|---|---|---|---|---|---|---|---|
| | | | Position | | variation | | Candidate |
| Phenotype | Locus ID | SV Type | (Mb)^{a} | β (se)^{†} | explained | p-value^{b} | Gene |
| ABS | Cfa6.66 | Deletion | 5.75 | 0.23 (0.09) | 4.45 | 1.38 × 10⁻⁴ | GTF2I |
| | Cfa6.3 | Insertion | 2.14 | 0.11 (0.07) | 0.56 | 8.12 × 10⁻⁴ | WBSCR17 |
| | Cfa6.7 | Deletion | 2.54 | 0.12 (0.07) | 0.62 | 8.89 × 10⁻⁴ | WBSCR17 |
| SIS | Cfa6.66 | Deletion | 5.75 | −27.0 (24.80) | 11.45 | 1.95 × 10⁻⁴ | GTF2I |
| Top three | Cfa6.72 | Deletion | 5.90 | 0.04 (0.052), | NA | 4.98 × 10⁻⁴ | GTF2IRD1 |
| principal | | | | −0.96 (0.57), | | | |
| components | | | | 1.11 (0.36) | | | |
| (PC model) | | | | | | | |

^{a}See Table 6 for locus details.
^{b}P-values from likelihood ratio test (Adjusted significance threshold p = 2.38 × 10⁻³).
^{†}β, effect size; se, standard error.
NA, Not applicable

In addition, two intergenic SVs were significantly associated with ABS (Cfa6.69, p=1.56×10⁻⁴; Cfa6.27, p=3.31×10⁻⁴), and Cfa6.27 was also associated with the PCs (p=1.24×10⁻⁴). However, the analyses were focused on genic SVs to infer any potential functional impact. Cfa6.66 was associated with multiple sociability metrics (ABS and SIS) and had the strongest two association signals (p=1.38×10⁻⁴ and p=1.95×10⁻⁴, respectively) (Table 9). GTF2I and GTF2IRD1 are members of the TFII-I family of transcription factors, a set of paralogous genes which have been repeatedly linked to the expression of hyper-sociability in mice (35,36), and are specifically implicated in the hyper-sociable phenotype of persons with WBS (37,38).

To disentangle the association of SVs with behavior from an association with species membership, species was incorporated as a covariate (Table 10).

TABLE 10
Genic loci associated with indices of human-directed social behavior
across dogs and wolves after inclusion of species as a covariate.

| | | | | | % | | |
|---|---|---|---|---|---|---|---|
| | Locus | SV | Position | | variation | | Candidate |
| Phenotype | ID | Type | (Mb) | β (se) | explained | p-value | Gene |
| ABS | Cfa6.66 | Deletion | 5.75 | 0.23 (0.091) | 5.76 | 2.33 × 10⁻⁴ | GTF2I |
| | Cfa6.7 | Deletion | 2.54 | 0.10 (0.081) | 0.58 | 9.56 × 10⁻⁴ | WBSCR17 |
| | Cfa6.3 | Insertion | 2.14 | 0.081 (0.076) | 0.50 | 1.06 × 10⁻³ | WBSCR17 |
| SIS | Cfa6.66 | Deletion | 5.75 | −9.7 (32) | 1.80 | 1.67 × 10⁻³ | GTF2I |

These analyses were consistent with the initial findings for Cfa6.66, Cfa6.3 and Cfa6.7. Locus Cfa6.66 remained significantly associated with multiple sociability metrics (ABS, p=2.33×10⁻⁴; SIS, p=1.67×10⁻³) and showed the strongest association of any genic SV. Cfa6.3 and Cfa6.7 both retained their associations with ABS (p=1.06×10⁻³ and p=9.56×10⁻⁴, respectively), as did the intergenic SVs Cfa6.69 (p=1.36×10⁻⁴) and Cfa6.27 (p=5.56×10⁻⁴). Furthermore, the ABS effect size (β) remained stable for the association models with and without species membership as a covariate (ABS β without covariates: Cfa6.3=0.11, Cfa6.7=0.12, Cfa6.27=−0.15, Cfa6.66=−0.23, Cfa6.69=−0.15; ABS 3 with covariates: Cfa6.3=0.081, Cfa6.7=0.10; Cfa6.27=−0.13; Cfa6.66=−0.23; Cfa6.69=−0.14), indicating that the observed effects on sociability are not an artifact of species differences. An association test of each locus with species membership further supports this interpretation as none of the behavior-associated SVs significantly associated with species membership alone (Table 11).

TABLE 11
Association to species membership.

| Locus ID | χ² | p-value | Odds Ratio |
|---|---|---|---|
| Cfa6.3 | 0.3345 | 0.563 | 1.615 |
| Cfa6.6 | 16.39 | 0.00005155 | NA |
| Cfa6.7 | 3.409 | 0.06484 | 7.154 |
| Cfa6.8 | 16.39 | 0.00005155 | NA |
| Cfa6.9 | 7.714 | 0.005479 | 14.09 |
| Cfa6.17 | 2.182 | 0.1396 | 0 |
| Cfa6.20 | 0.08362 | 0.7724 | 0.7714 |
| Cfa6.22 | 0.4465 | 0.504 | 0.4667 |
| Cfa6.24 | 2.791 | 0.09481 | 0 |
| Cfa6.25 | 1.172 | 0.279 | 1.988 |
| Cfa6.26 | 0.6969 | 0.4038 | 0.5844 |
| Cfa6.27 | 6.4 | 0.01141 | NA |
| Cfa6.28 | 0.8571 | 0.3545 | 0.36 |
| Cfa6.29 | 0.2359 | 0.6272 | 0.6923 |
| Cfa6.32 | 0.04356 | 0.8347 | 0.8769 |
| Cfa6.35 | 2.462 | 0.1167 | 3.182 |
| Cfa6.40 | 0.6154 | 0.4328 | 1.8 |
| Cfa6.42 | 3.429 | 0.06408 | 5 |
| Cfa6.44 | 0.1678 | 0.682 | 1.286 |
| Cfa6.47 | 5.897 | 0.01517 | 5.444 |
| Cfa6.50 | 3.376 | 0.06616 | 3.37 |
| Cfa6.54 | 0.5 | 0.4795 | 1.623 |
| Cfa6.56 | 0.6154 | 0.4328 | 1.8 |
| Cfa6.57 | 13.71 | 0.0002128 | NA |
| Cfa6.59 | 0.04196 | 0.8377 | 0.8815 |
| Cfa6.60 | 0.06316 | 0.8016 | 0.8242 |
| Cfa6.66 | 4.5 | 0.03389 | 0.1273 |
| Cfa6.68 | 0.9435 | 0.3314 | 1.97 |
| Cfa6.69 | 0.6154 | 0.4328 | 1.8 |
| Cfa6.72 | 0.1364 | 0.7119 | 0.6444 |
| Cfa6.75 | 0.5455 | 0.4602 | 2.143 |
| Cfa6.77 | 2.889 | 0.08916 | 3.24 |
| Cfa6.78 | 0.3345 | 0.563 | 1.615 |
| Cfa6.79 | 0.3345 | 0.563 | 1.615 |
| Cfa6.82 | 6.4 | 0.01141 | NA |
| Cfa6.83 | 2.091 | 0.1482 | 3.222 |
| Cfa6.86 | 0.4465 | 0.504 | 0.4667 |

### Example 4—Functional Impact of Annotated Structural Variants

It was next determined whether these behavior-associated SVs were predicted to have a functional impact. Ensembl's Variant Effect Predictor (VEP) v84 (39) was used with Ensembl transcripts for the CanFam 3.1 reference genome to assign putative functional consequences to all insertions, deletions, and duplications in the filtered set of SVs. Due to a software limitation that VEP is unable to assign consequences for transitions, inversions, and complex SV, seven sites (6 TRA, 1 INV, 1 D_I) in the UCSC genome browser were manually inspected with Ensembl gene models (40). Three transcription ablations, seven loss-of-start codons, and five transcript amplifications (Table 12) were found.

TABLE 12
Predicted Functional Consequences of SVs. Consequences
predicted using Ensembl Variant Effect Predictor.

| | | IMPACT | # of |
|---|---|---|---|
| Consequence | Description | rating | SVs |
| Transcript ablation | Deleted region includes | High | 3 |
| | a transcript feature | | |
| Start lost | Changes at least one base | High | 7 |
| | of start codon | | |
| Transcript amplification | Amplification of region | High | 5 |
| | containing a transcript | | |
| Coding sequence variant | Changes the coding | Modifier | 10 |
| | sequence of a gene | | |
| Feature truncation | Reduces genomic feature | Modifier | 16 |
| | relative to reference | | |
| Feature elongation | Located within a regulatory | Modifier | 12 |
| | region | | |
| Non-coding transcript | Transcript variant of a | Modifier | 3 |
| variant | non-coding RNA gene | | |
| Intronic variant | Located in an intron | Modifier | 32 |
| 5′ UTR variant | Located in 5′ UTR | Modifier | 8 |
| 3′ UTR variant | Located in 3′ UTR | Modifier | 1 |
| Upstream variant | Located 5′ of a gene | Modifier | 9 |
| Downstream variant | Located 3′ of a gene | Modifier | 4 |
| Intergenic variant | Located >5 kb from a gene | Modifier | 40 |

All SVs significantly associated with human-directed social behavior were ‘feature truncations’, except for Cfa6.3, which was a ‘feature elongation’ that likely is due to a lost stop codon or the elongation of an internal sequence feature relative to the reference. Annotation of Cfa6.3, Cfa6.7, Cfa6.66 and Cfa6.72 as modifiers of gene function suggests a direct association between these variants and human-directed social behavior as quantified by behavioral measures, mediated by possible interference with WBSCR17, GTF2I and GTF2IRD1.

### Example 5—PCR Validation and Analysis of Structural Variants

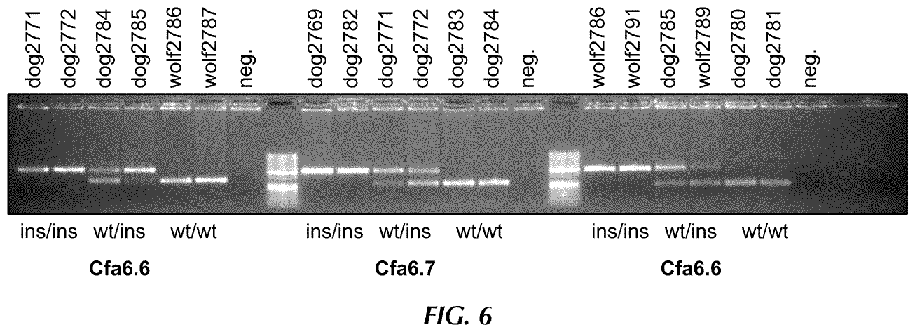

The in silico SV detection algorithms applied to the targeted resequencing data can identify the presence or absence of an SV, but cannot predict the underlying genotype of an individual for a given SV. To corroborate the in silico findings and investigate the possibility of other genetic models, PCR amplification and agarose gel electrophoresis were used to determine the codominant genotypes at the top four loci (Cfa6.6, Cfa6.7, Cfa6.66, and Cfa6.83) (FIG. 6). These four SVs overlapped with short interspersed nuclear transposable elements (TEs) with high sequence identity to the reference (182 to 259 bp, 91-96% pairwise identity over 193 bp). Insertional variation in 298 canids consisting of coyotes, gray wolves (representing populations from Europe, Asia, and North America), AKC-registered breeds, and semi-domestic dog populations (see Methods) were further surveyed. The analysis was repeated with the co-dominant SV genotypes to determine if there was an associated with species membership. Coyotes were excluded from this analysis, and semi-domestic dogs were grouped with domestic dog.

All outlier SVs, now with co-dominant genotypes, were significantly associated with species membership (Cfa6.6 χ²=23.91, p=1.01×10⁻⁶, OR=0.33; Cfa6.7 χ²=57.63, p=³0.16×10⁻¹⁴, OR=13.83; Cfa6.66 χ²=35.12, p=3.1×10⁻⁹, OR=0.25; Cfa6.83 χ²=17.11, p=3.53×10⁻⁵, OR=NA), confirming this region's original identification (19). Similar results were obtained if “modern” breeds only were included, as per the original method that located this region (19) (Cfa6.6 χ²=11.9, p=0.0006, OR=0.45; Cfa6.7 χ²=40.87, p=1.63×10⁻¹⁰, OR=10.35; Cfa6.66 χ⁻⁼41.97, p=9.25×10⁻¹¹, OR=0.20; Cfa6.83 χ²=20.41, p=6.24×10⁻⁶, OR=NA), with site-specific patterns (frequency of TE insertion in modern dogs and wolves, respectively: Cfa6.6=−0.52 and 0.32; Cfa6.7=0.39 and 0.06; Cfa6.66=0.10 and 0.37; Cfa6.83=0.17 and 0.00).

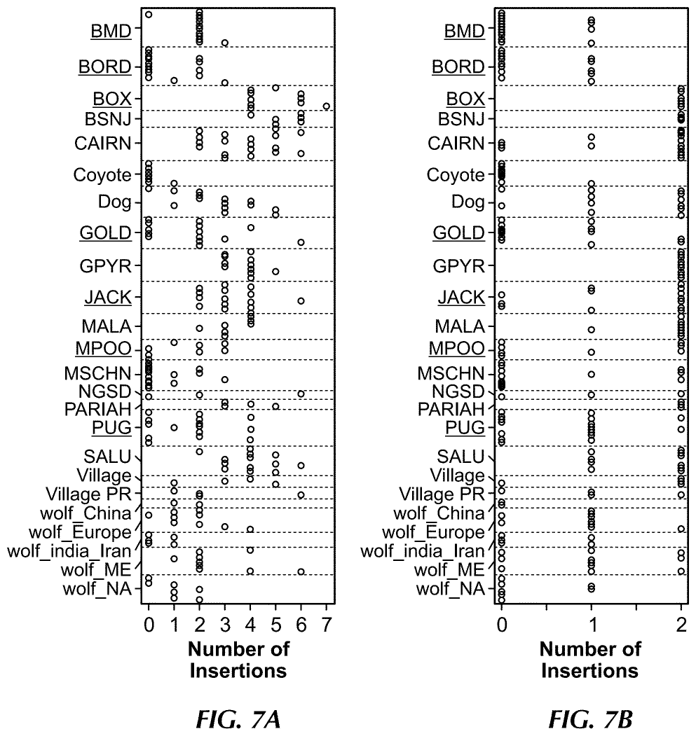

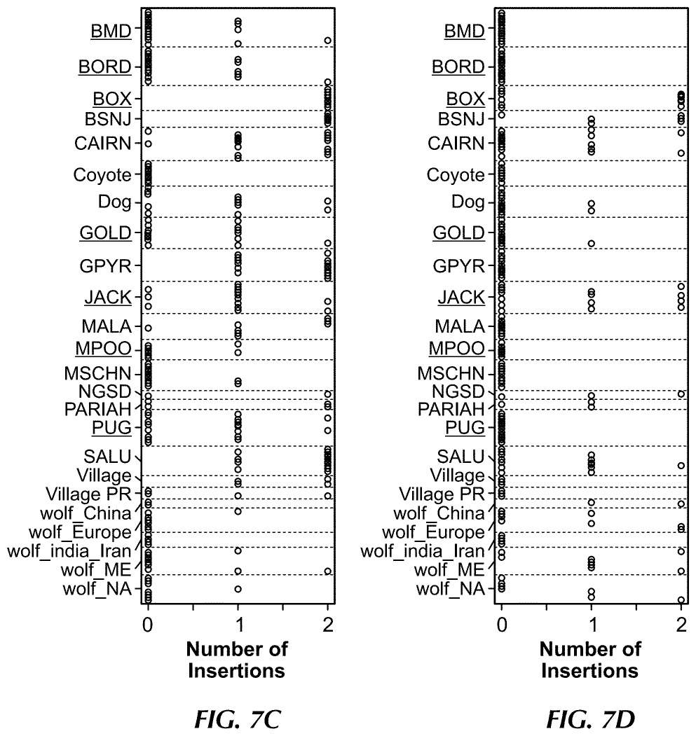

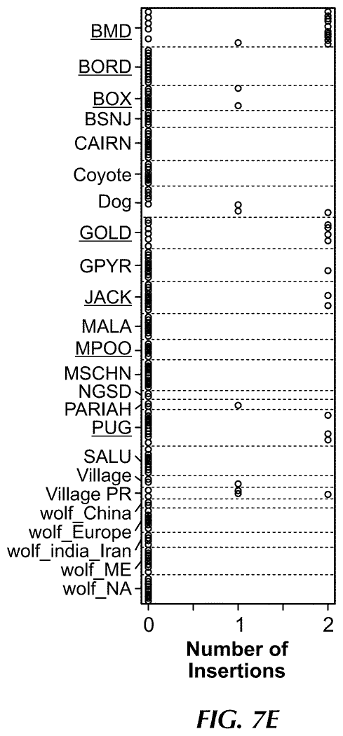

The frequency of insertions per locus by population or species membership was calculated. The TEs segregated at low frequencies in coyotes and were variable across wolf populations and dog breeds (FIG. 7). Only one coyote carried a single insertion of the TE at locus Cfa6.6, with both Cfa6.6 and Cfa6.7 highly polymorphic across domestic dogs (FIGS. 7B,C). Locus Cfa6.66 is found in wolves from China, Europe, the Middle East, and the WBS study wolves, but only within six dog breeds (Boxer, Basenji, Cairn terrier, Golden retriever, Jack Russell terrier, and Saluki), the WBS dogs, two NGSDs, and a single Pariah dog (FIG. 7D). Cfa6.83 appears to be a de novo insertion within domestic dogs as it is lacking entirely within the wild canids (FIG. 7E), with a low to moderate frequency within the semi-domestic dog populations surveyed (n, Pariah dog=1; n, Village dogs: Africa=1, Puerto Rico=5). Based on the WBS dogs and wolves with behavioral data, trends per locus were noted as: more insertions at Cfa6.6 were correlated with increased ABS and HYP (r=0.50 and 0.42, respectively), with weaker relationships for SIS (r=0.11); more insertions at Cfa6.7 correlated with increased ABS and HYP, with an inverse relationship with SIS (r=0.13, 0.11, and −0.17, respectively); fewer insertions at Cfa6.66 is correlated with higher trait values (r=−0.59, −0.56, and −0.27, for ABS, HYP, and SIS respectively); more insertions at Cfa6.83 increased all behavioral trait values (r=0.36, 0.44, and 0.40, for ABS, HYP, and SIS respectively).

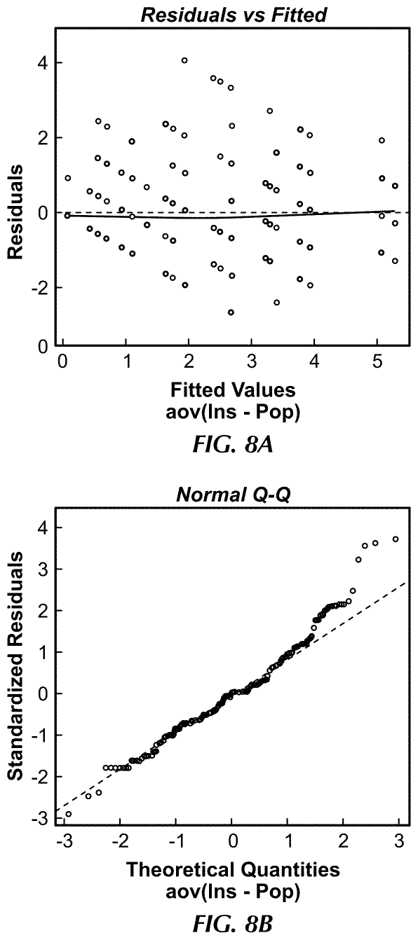

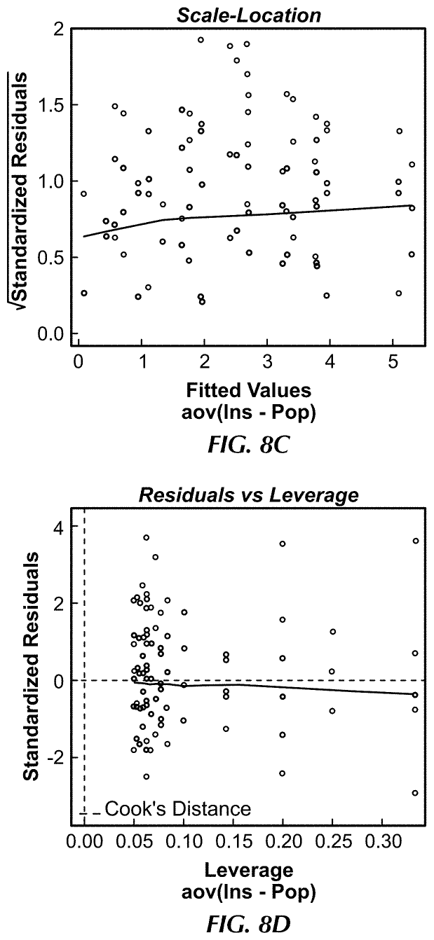

One-way ANOVA was conducted using the population or species designation as a predictor of the total number of insertions across four outlier loci. The total number of insertions significantly depends on the population (F(23,274)=19.54; p<2×10⁻¹⁶), with 103 of 276 pairwise population mean comparisons contributing to the ANOVA significance (dog/dog=46, wolf/dog=28, coyote/dog=11, semi-domestic/dog=8, semi-domestic/coyote=3, semi-domestic/wolf=3, wolf/coy=2, and wolf/wolf=2; Tukey HSD, p<0.05) (FIG. 8).

As the gel-based genotyping method now reveals a co-dominant genotype compared to the in silico status, an association scan was conducted for each of the four outlier SV loci with the binary phenotype for each AKC breed (41), village dogs and pariah dogs as “Seeks attention” or “Avoids attention” using two logistic regression models in R, an additive and dominant model, with sex as a covariate. The use of breed-based stereotypes is supported by the strict genetic isolation and selective breeding efforts that maintain breeds. As such, many traits strongly determined by genetic variation (including behavioral) can be predicted with high accuracy. The central foundation and advantage of domestication and breed formation is that selection for many traits, including behavior, has been very strong and, thus, the number of underlying genes is apt to be small. As a proof of principal, Jones et al. successfully mapped a variety of breed-associated traits in a genome-wide association study using dog “stereotypes” (9). They scored breeds for pointing, herding, boldness, and trainability, and identified one locus associated to pointing, three for herding, and one for trainability. Most importantly, they found five for boldness. These loci contain likely candidate genes, many of which are important in schizophrenia, dopamine receptors, and proteins linked to synaptic junctions. Vaysse et al. (16) also utilized breed stereotypes to map behaviors, such as boldness, sociability, curiosity, playfulness, chase-proneness, and aggressiveness. They mapped boldness to an intron of HMGA2, and sociability, defined as the “dog's attitude towards unknown people”, to a gene on the X chromosome, after excluding male dogs from the analysis to accurately compare autosomal and sex-chromosome patterns of genetic variation.

Significant support was found for an association between three of the four loci and the binary behavioral trait of seeking or avoiding attention (additive model: Cfa6.6 OR=0.303 p=2.79×10⁻¹⁰, Cfa6.7 OR=0.398 p=4.66×10⁻⁷, Cfa6.83 OR=2.95 p=2.83×10⁻⁴; dominant model: Cfa6.6 OR=0.184 p=8.22×10⁻⁷, Cfa6.7 OR=0.287 p=4.31×10⁻⁵, Cfa6.83 OR=5.04 p=6.50×10⁻⁴; sex was not a significant predictor in any of these models). SV Cfa6.66 was not significant (additive model: OR=0.852 p=0.496; dominant model: OR=0.573 p=0.124). Further, logistic regression found that TE copy number could significantly predict the binary breed stereotype behavior of attention seeking or avoidance (OR=0.676 per insertion, p=1.13×10⁻⁵ with no evidence of a sex effect).

### Example 6—Genome-Wide SNP Survey

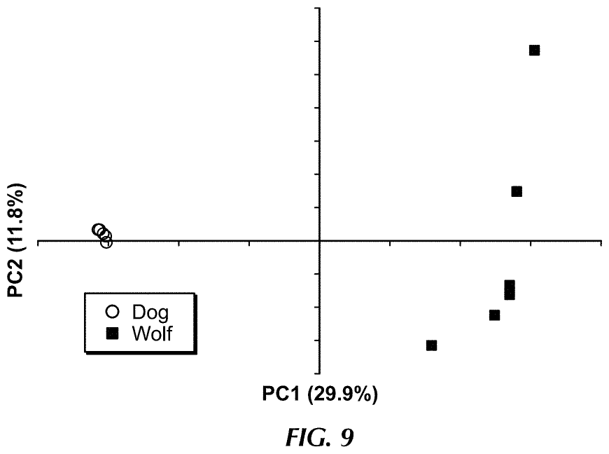

To identify additional candidate loci, genome-wide SNP genotypes were collected using the Affymetrix Axiom K9HDSNPA (643,641 loci) and Axiom K9HDSNPB (625,577 loci) arrays. A PCA was conducted on 544K genome-wide SNP genotypes to ensure the expected spatial clustering pattern of the samples. With a subset of 25,510 uncorrelated and unlinked SNPs, a PCA confirmed the discrete spatial separation of the two species (PC1, 29.9%; PC2, 11.8%) (FIG. 9). This finding was further supported by high average genome-wide differentiation (F_{ST}=0.194), a level comparable to the original finding (19). Next, a binary association test was conducted on species membership in GEMMA and found support for the candidate locus WBSCR17 as containing species-specific variation (p<3×10⁻⁶). Further, each of the quantitative behavioral indices (ABS, HYP, SIS) was tested in a univariate regression analysis on the 544K SNP set and identified 222 additional SNPs within the 5 Mb target region associated with two behavioral traits (HYP: n_{SNP}_{ₛ}=84, mean p=0.002; SIS: n_{SNP}_{ₛ}=138, mean p=0.001). The quantitative association testing identified 77,889 SNPs outside of the resequenced region associated with each behavioral trait (n SNPs: ABS=874, HYP=19373, SIS=57642, p<0.005), implicating 221 genes associated with ABS, 3520 genes with HYP, and 3118 genes with SIS. Of these, only a single gene ontology term associated with ABS (Phosphoric ester hydrolase activity), 30 terms with HYP, and 26 with SIS (Tables 13, 14).

TABLE 13
The significantly enriched (adjusted p < 0.05) gene ontology term from a
quantitative association test with each behavioral trait and 544K genome-wide SNPs.

| Behavioral | Data | | | | | | | Adjusted |
|---|---|---|---|---|---|---|---|---|
| trait | base | Name | C | O | E | R | p-value | p-value |
| ABS | MF | Phosphoric ester hydrolase activity | 203 | 10 | 2.58 | 3.87 | 0.0003 | 0.0483 |
| HYP | BP | Cell development | 899 | 208 | 136 | 1.53 | 1.47⁻¹¹ | 5.61⁻⁸ |
| | BP | Generation of neurons | 575 | 144 | 87 | 1.66 | 9.84⁻¹¹ | 1.25⁻⁷ |
| | BP | Cellular developmental process | 1679 | 342 | 254 | 1.36 | 8.42⁻¹¹ | 1.27⁻⁷ |
| | BP | System development | 2098 | 410 | 317.4 | 1.29 | 2.27⁻¹⁰ | 1.44⁻⁷ |
| | BP | Cell differentiation | 1556 | 319 | 235.4 | 1.36 | 2.16⁻¹⁰ | 1.44⁻⁷ |
| | BP | Anatomical structure development | 2445 | 467 | 369.9 | 1.26 | 2.22⁻¹⁰ | 1.44⁻⁷ |
| | BP | Neuron differentiation | 506 | 129 | 77 | 1.68 | 4.46⁻¹⁰ | 2.13⁻⁷ |
| | BP | Nervous system development | 892 | 201 | 134.9 | 1.49 | 4.14⁻¹⁰ | 2.13⁻⁷ |
| | BP | Neurogenesis | 626 | 151 | 94.7 | 1.59 | 6.25⁻¹⁰ | 2.65⁻⁷ |
| | BP | Multicellular organismal | 2298 | 439 | 347.6 | 1.26 | 1.11⁻⁹ | 4.24⁻⁷ |
| | | development | | | | | | |
| | MF | GTPase regulatory activity | 167 | 51 | 25.14 | 2.03 | 2.49⁻⁷ | 6.71⁻⁵ |
| | MF | Small GTPase regulatory activity | 123 | 41 | 18.5 | 2.21 | 2.83⁻⁷ | 6.71⁻⁵ |
| | MF | Protein binding | 5688 | 948 | 856.4 | 1.11 | 1.10⁻⁷ | 6.71⁻⁵ |
| | MF | Phosphoric ester hydrolase activity | 203 | 58 | 30.6 | 1.9 | 4.78⁻⁷ | 8.5⁻⁵ |
| | MF | Nucleoside-triphosphatase | 172 | 51 | 25.9 | 1.97 | 6.85⁻⁷ | 9.74⁻⁵ |
| | | regulatory activity | | | | | | |
| | MF | Guanyl-nucleotide exchange factor | 77 | 27 | 11.6 | 2.33 | 1.04⁻⁵ | 0.0009 |
| | | activity | | | | | | |
| | MF | Ion channel activity | 242 | 62 | 36.43 | 1.7 | 1.05⁻⁵ | 0.0009 |
| | MF | Phospholipid binding | 225 | 59 | 33.9 | 1.74 | 7.92⁻⁶ | 0.0009 |
| | MF | Substrate-specific channel activity | 246 | 62 | 37 | 1.67 | 1.81⁻⁵ | 0.0013 |
| | MF | Binding | 7867 | 1244 | 1184.4 | 1.05 | 1.86⁻⁵ | 0.0013 |
| | CC | Synapse | 180 | 60 | 27.1 | 2.21 | 5.04⁻¹⁰ | 2.22⁻⁷ |
| | CC | Cell periphery | 1588 | 314 | 239.1 | 1.31 | 1.32⁻⁸ | 1.94⁻⁶ |
| | CC | Cell projection | 569 | 135 | 85.7 | 1.58 | 1.27⁻⁸ | 1.94⁻⁶ |
| | CC | Plasma membrane | 1512 | 298 | 227.7 | 1.31 | 5.01⁻⁸ | 5.51⁻⁶ |
| | CC | Neuron projection | 246 | 68 | 37 | 1.84 | 1.96⁻⁷ | 1.72⁻⁵ |
| | CC | Proteinaceous extracellular matrix | 169 | 51 | 25.5 | 2 | 3.72⁻⁷ | 2.73⁻⁵ |
| | CC | Axon | 123 | 38 | 18.5 | 2.05 | 6.09⁻⁶ | 0.0003 |
| | CC | Extracellular matrix | 243 | 63 | 36.7 | 1.72 | 5.80⁻⁶ | 0.0003 |
| | CC | Basement membrane | 65 | 24 | 9.8 | 2.45 | 1.17⁻⁵ | 0.0006 |
| | CC | Dendrite | 99 | 31 | 14.9 | 2.08 | 3.19⁻⁵ | 0.0014 |
| SIS | BP | Cell development | 899 | 215 | 150.8 | 1.43 | 4.72⁻⁹ | 1.80⁻⁵ |
| | BP | Cell adhesion | 429 | 113 | 72 | 1.57 | 1.99⁻⁷ | 0.0003 |
| | BP | Biological adhesion | 430 | 113 | 72.1 | 1.57 | 2.27⁻⁷ | 0.0003 |
| | BP | Transmission of nerve impulse | 266 | 76 | 44.6 | 1.7 | 7.74⁻⁷ | 0.0004 |
| | BP | Multicellular organismal signaling | 269 | 77 | 45.1 | 1.71 | 6.04⁻⁷ | 0.0004 |
| | BP | Single-organism behavior | 229 | 68 | 38.4 | 1.77 | 6.44⁻⁷ | 0.0004 |
| | BP | Nervous system development | 892 | 203 | 149.6 | 1.36 | 7.46⁻⁷ | 0.0004 |
| | BP | Neurogenesis | 626 | 147 | 105 | 1.4 | 5.02⁻⁶ | 0.0021 |
| | BP | Cellular developmental process | 1679 | 345 | 281.7 | 1.22 | 4.31⁻⁶ | 0.0021 |
| | BP | Neuron differentiation | 509 | 123 | 85.4 | 1.44 | 7.32⁻⁶ | 0.0026 |
| | MF | Protein binding | 5688 | 1086 | 977.7 | 1.11 | 3.16⁻⁹ | 2.42⁻⁶ |
| | MF | Binding | 7867 | 1432 | 1352.3 | 1.06 | 7.2⁻⁸ | 2.76⁻⁵ |
| | MF | Kinase activity | 396 | 96 | 68 | 1.41 | 0.0002 | 0.0383 |
| | MF | Peptide hormone binding | 7 | 6 | 1.2 | 4.99 | 0.0002 | 0.0383 |
| | MF | Protein tyrosine kinase activity | 81 | 27 | 13.9 | 1.94 | 0.0003 | 0.0383 |
| | MF | Calcium-release channel activity | 10 | 7 | 1.7 | 4.07 | 0.0003 | 0.0383 |
| | CC | Neuron projection | 246 | 77 | 41.2 | 1.87 | 8.93⁻⁹ | 4.07⁻⁹ |
| | CC | Cell projection | 569 | 141 | 95.3 | 1.48 | 2.97⁻⁷ | 6.77⁻⁵ |
| | CC | Synapse | 180 | 57 | 30.1 | 1.89 | 4.99⁻⁷ | 7.58⁻⁵ |
| | CC | Basement membrane | 65 | 27 | 10.9 | 2.48 | 1.88⁻⁶ | 0.0002 |
| | CC | Proteinaceous extracellular matrix | 169 | 51 | 28.3 | 1.8 | 9.27⁻⁶ | 0.0008 |
| | CC | Axon | 123 | 40 | 20.6 | 1.94 | 1.22⁻⁵ | 0.0009 |
| | CC | Dendrite | 99 | 33 | 16.6 | 1.99 | 3.94⁻⁵ | 0.0026 |
| | CC | Cell periphery | 1588 | 320 | 265.9 | 1.2 | 5.25⁻⁵ | 0.003 |
| | CC | Extracellular matrix | 243 | 62 | 40.7 | 1.52 | 0.0003 | 0.0152 |
| | CC | Plasma membrane | 1512 | 299 | 253.2 | 1.18 | 0.0004 | 0.0182 |

Abbreviations: biological process, BP; number of reference genes in the category, C; expected number in category, E; molecular function, MF; number of genes in the gene set and category, O; ratio of enrichment, R.

TABLE 14
The significantly enriched (adjusted p < 0.05) gene ontology term from
the univariate regression analysis conducted in GEMMA with each behavioral
trait and 544K genome-wide SNPs. HYP had no significant GO categories enriched.

| Behavioral | | | | | | | p- | Adjusted |
|---|---|---|---|---|---|---|---|---|
| trait | Database | Name | C | O | E | R | value | p-value |
| ABS | BP | Regulation of neuron maturation | 2 | 2 | 0.02 | 97.14 | 0.0001 | 0.0377 |
| | BP | Negative regulation of neuron | 2 | 2 | 0.02 | 97.14 | 0.0001 | 0.0377 |
| | | maturation | | | | | | |
| | CC | Synapse | 180 | 8 | 1.79 | 4.47 | 0.0004 | 0.0400 |
| SIS | CC | Cell periphery | 1588 | 30 | 15.10 | 1.99 | 8.88⁻⁵ | 0.0090 |
| | CC | Plasma membrane | 1512 | 28 | 14.38 | 1.95 | 0.0002 | 0.0101 |
| | CC | Cell junction | 309 | 10 | 2.94 | 3.40 | 0.0007 | 0.0236 |

Abbreviations: biological process, BP; number of reference genes in the category, C; expected number in category, E; molecular function, MF; number of genes in the gene set and category, O; ratio of enrichment, R.

### Example 7—Behavioral Data

Dogs and wolves were ensured to be in the same developmental stage by only including subjects over one year of age, well past the species-specific window for primary socialization. All dogs and wolves were socialized to humans as puppies, received daily contact from human caretakers, and experienced regular free-contact interactions with unfamiliar humans from puppyhood through the time of this study. To ensure the wolves used in this study had been socialized to accepted standards and were as familiar to their caretakers as possible, wolves were only included if they had been hand-reared by humans from before 10-14 days of age following the procedures established by Klinghammer & Goodman (70), and were still living in the same facility in which they were raised. Wolves experienced 24-hour contact with human caretakers for at least the first six weeks of life, followed by contact during daylight hours until four months of age and then daily human interaction with caretakers and other humans thereafter. Therefore, in the current study, the lower level of sociability displayed towards familiar individuals by wolves in comparison to pet dogs could not be explained by lack of initial bond formation (socialization) or insufficient familiarity with their caretakers. In fact, wolves did show social interest in their caretakers, approaching them for greetings when they entered during the sociability test in this study. However, they then returned to other activities. This pattern of behavior might be considered a ‘typical’ social greeting for bonded adult animals, whereas the prolonged greeting of pet dogs, sometimes lasting the full two minutes, would be considered exaggerated or hyper-social (7).

To ensure equivalent testing conditions each species was tested in a controlled setting most constant with their home environment (71). Dogs were individually tested at an indoor location in Corvallis Oreg., USA; wolves were tested in a familiar outdoor enclosure at Wolf Park, Battle Ground Indiana, USA. Testing procedures were the same for both species. Each subject was assessed using two tests designed to quantitatively probe their human-directed sociability along indices relevant to the clinical presentation of WBS: a solvable task test and a sociability test (7,8). Data from the solvable task test and sociability test were used to calculate three indices relevant to behaviors that typify WBS in humans: attentional bias to social stimuli (ABS), hyper-sociability (HYP), and social interest in strangers (SIS) (Table 15).

TABLE 15
Behavioral data and description relative to WBS.

| | Quantified task and | | Refer- |
|---|---|---|---|
| Behavior | information on WBS | Calculation | ence |
| Attentional | Higher proportion of | The ratio of the | 22 |
| bias towards | time referencing | proportion of time | |
| social stimuli | familiar human | spent looking at the | |
| (ABS) | Lower proportion of | experimenter to the | |
| | time looking at object | sum of the proportion of | |
| | Lower proportion of | time spent looking at the | |
| | time physically | experimenter plus the | |
| | contacting object | proportion of time spent | |
| | | looking at the puzzle box | |
| | | in the solvable task test. | |
| Hyper- | Higher proportion of | Sum of the time spent in | 22, 37 |
| sociability | time spent in | proximity to the | |
| (HYP) | proximity of | experimenter in each | |
| | familiar or | phase of the sociability | |
| | unfamiliar human | test. | |
| Social | Higher proportion of | Sum of the time spent in | 22, 37 |
| interest in | time in proximity | proximity to the | |
| strangers | with unfamiliar human | experimenter in the two | |
| (SIS) | | unfamiliar phases of the | |
| | | sociability test. | |

Those tests are described in detail in the following sections.

Solvable Tasks and Sociability Measures.

The solvable task test was used to measure individual problem solving performance, attentiveness to humans and the degree to which a familiar human's presence interfered with independent problem solving behavior. Although this problem-solving task is considered challenging, it has previously been validated as physically solvable by wolves, small dogs, and large dogs (8). All subjects were naïve to the problem prior to testing and humans were instructed to remain passive and neutral after placing the container on the ground.

The sociability test consisted of a passive and an active phase, each lasting two minutes. One wolf (ID 2794) was not available for sociability testing, therefore sociability analysis was conducted on all 18 dogs and 9 wolves. The experimenter spoke to and touched the subject if the animal came close enough to reach while remaining on the bucket or chair. If the animal moved away, then the experimenter called his/her name again to regain the subject's attention. All trials were recorded on video. For each condition, videos were coded for time spent in proximity to the experimenter, and time spent touching the experimenter (27). An independent coder blind to the purpose of this study double coded 42% of these videos; inter-rater reliability was determined to be strong using a weighted Cohen's kappa, K=0.75 (95% confidence interval: 0.64-0.86) (72).

It should be noted that many of the wolves in the current study have participated and performed as well as or better than pet domestic dogs on tasks related to social cognition (using human cues to solve problems) (26). In the current study they quickly approached the humans to initiate a greeting or to receive the puzzle box. The key difference observed was that adult dogs were more likely to engage in prolonged or exaggerated contact with humans than adult wolves.

Behavioral Indices Relevant to WBS in Humans.

Data from the solvable task test and the sociability test were used to quantify canine behavior along indices relevant to the sociable phenotype of WBS including: 1) time spent looking at the puzzle box in the solvable task test (“time look box”), 2) time spent looking at the human in the solvable task test (“time look human”), time spent in proximity to a familiar experimenter in the 3) active and 4) passive phases of the sociability test (“proximity familiar active” and “proximity familiar passive”), and time spent in proximity to an unfamiliar experimenter in the 5) active and 6) passive phases of the sociability test (“proximity unfamiliar active” and “proximity unfamiliar passive”).

Data from the solvable task test and sociability test were used to calculate three indices relevant to the behavior under selection during dog domestication and analogous to behaviors that typify WBS in humans: attentional bias to social stimuli (ABS), hyper-sociability (HYP), and social interest in strangers (SIS). ABS was calculated as the ratio of time spent looking at the experimenter to the sum of the time spent looking at the experimenter and the time spent looking at the puzzle box in the solvable task test and was intended to quantify the proportion of the animal's attention directed towards the experimenter. HYP was calculated as the sum of the time spent in proximity to the experimenter in each phase of the sociability test and was intended to quantify engagement with humans across social scenarios. SIS was calculated as the sum of the time spent in proximity to the experimenter in the two unfamiliar phases of the sociability test and was intended to quantify engagement with unfamiliar persons (Tables 2, 15).

Principal Components Analysis of Behavioral Indices.

Dog and wolf behavior was also characterized by principal components analysis using data from the Solvable Task Test (8) and Sociability Test (73) (Table 2) with the prcomp function in R (http://www.r-project.org/).

Inclusion of PCs was assessed with the nFactors package in R (74). The majority of component retention analyses indicated inclusion of the top two principal components (Kaiser's Rule: 2, Horn's parallel analysis: 2, acceleration factor: 2, optimal coordinates: 1). However, it was found a relatively low percentage of behavioral variation was explained by the first two principal components (cumulatively, 72%) and a lack of an obvious knee in the scree plot (FIG. 4). Additionally, previous research has shown that inclusion of a greater number of phenotypic principal components significantly increases the power of genome-wide associations (75). Therefore, the top three PCs were selected for use as phenotypes in regression analyses.

### Example 8—Genetic Sample Collection and Genomic Enrichment

Following behavioral trials, 2-3 ml of blood was collected from each dog and wolf from the cephalic, saphenous or jugular vein depending on the individual, temperament, and accessibility of the vein. Blood was deposited into a sterile blood collection tube, labeled, and then immediately placed in a freezer kept below −18 degrees Celsius until shipped overnight on ice for analysis. 24 out of 28 samples were chosen to sequence (n, dogs=16, wolves=8). Two of the original 18 dogs were removed from sequencing due to their low DNA yield; two of the original 10 wolves were excluded from sequencing due to the lack of an opportunity to redraw blood samples from these individuals, either due to our institutional protocols or due to the unavailability of the individual (Tables 1, 4). Genomic DNA was prepared from blood samples using QIAamp DNA mini kits (Qiagen, DNeasy blood and Tissue kit). DNA was quantified using a Qubit 2.0 Fluorometer and checked on a 2% agarose gel for degradation. A region under positive selection in the domestic dog genome on chromosome 6 that was identified from a genome-wide scan of 48,036 SNPs (19) was followed up on, through targeted resequencing of a ˜5 Mb contiguous block (2,031,491-7,215,670 bp) that contained 46 Ensembl-annotated genes (40,76), 27 of which have been described in WBS (Table 16).

TABLE 16
Genes in target region on canine chromosome 6 (CFA6).
Positions are from canfam3.1 genome build.

| Gene Start (bp) | Gene End (bp) | Gene Name | Reference |
|---|---|---|---|
| 2132919 | 2563654 | WBSCR17 | 19 |
| 2749188 | 2831960 | AUTS2 | 19 |
| 5606042 | 5632471 | WBSCR16 | 105 |
| 5700832 | 5719439 | NCF1 | 105 |
| 5722967 | 5811965 | GTF2I | 105 |
| 5885985 | 5963867 | GTF2IRD1 | 105 |
| 6028219 | 6090774 | CLIP2 | 105 |
| 6136604 | 6159026 | RFC2 | 105 |
| 6164647 | 6171316 | LAT2 | 105 |
| 6180064 | 6192330 | EIF4H | 105 |
| 6264548 | 6285910 | LIMK1 | 105 |
| 6304623 | 6321742 | ELN | 105 |
| 6472520 | 6474969 | WBSCR28 | 105 |
| 6488348 | 6492871 | WBSCR27 | 105 |
| 6493513 | 6494145 | CLDN4 | 105 |
| 6534229 | 6535237 | CLDN3 | 105 |
| 6550966 | 6556271 | ABHD11 | 105 |
| 6574691 | 6581692 | STX1A | 105 |
| 6595294 | 6595974 | DNAJC30 | 105 |
| 6602419 | 6606916 | VPS37D | 105 |
| 6633050 | 6652557 | MLXIPL | 105 |
| 6656046 | 6674950 | TBL2 | 105 |
| 6680478 | 6701631 | BCL7B | 105 |
| 6709697 | 6778186 | BAZ1B | 105 |
| 6782774 | 6784558 | FZD9 | 105 |
| 6836043 | 6868433 | FKBP6 | 105 |
| 6887596 | 6899406 | NSUN5 | 105 |

A full-service option offered by MYcroarray for DNA enrichment and genomic library preparation was used. 80mer bait probes to target the region of interest (MYbaits kit design) were designed. Genomic DNA was sonicated to approximately 300 bp fragment sizes, of which 500 ng were used to construct Illumina TruSeq sequencing libraries. Each library was dual-index-amplified for eight cycles of PCR, yielding between 590 ng and 1744 ng of the sequencing library. Of this, 500 ng was used for the target enrichment with a custom MYbaits kit. Following enrichment, libraries were amplified for six cycles, yielding between 6.7 ng and 14.7 ng of library. Libraries were standardized by pooling 5 ng from each library to a volume of 30 uL at 4 ng/uL for paired-end 2×67 nt sequencing in a single lane of Illumina HiSeq2500. Refer to Table 5 for enrichment summary statistics.

### Example 9—Sequence Data Processing and Bioinformatics

For strict deplexing, sequences with perfect matches between the observed and expected index sequence tags were retained. Reads were trimmed and clipped with cutadapt-1.8.1 (77) to discard reads that were <20 bp in length, exclude sites of low quality (<20), and remove remnant TruSeq adapter sequence. Mean and standard deviations of library insert sizes were calculated individually for each animal with a custom python script (https://gist.github.com/davidliwei/2323462). All reads were mapped to the unmasked reference dog chromosome 6 (CanFam3.1, Ensembl) generated from a boxer breed individual with BWA-0.7.12 (78). PCR duplicates were marked and removed with picard-tools-1.138 (http://picard.sourceforge.net). BAM files were then indexed, sorted, and VCF files produced from SAMtools (79), from which sequencing descriptive statistics were calculated. From the sorted BAM files, ANGSD (80) was used to call SNP genotypes with a minimum depth of 10× sequence coverage, a minimum mapping quality 30, SNP p<0.00001 and posterior probability>0.95, and a minimum variant quality of 20. Scores were also adjusted around insertions/deletions with the -baq flag. Monomorphic sites were excluded.

SNP genotypes were phased with SHAPEIT (81). The region was scanned for signals of positive selection in the dog genome using cross population extended haplotype homozygosity (XP-EHH [82]) of 4,844 SNPs within the resequenced region. Per-SNP F_{ST} was calculated with a custom script (19). Both the F_{ST} and XP-EHH scores were normalized into a z-score to have a mean of zero and standard deviation of 1. The product of their z-scores represented their composite “bivariate percentile score”. The empirical rule was used to identify outlier loci in the 97.5^{th} percentile or greater (z score>2). Peaks of selection had to contain at least three outlier loci to be considered.

### Example 10—De Novo Annotation and Genotype Calling of Structural Variants

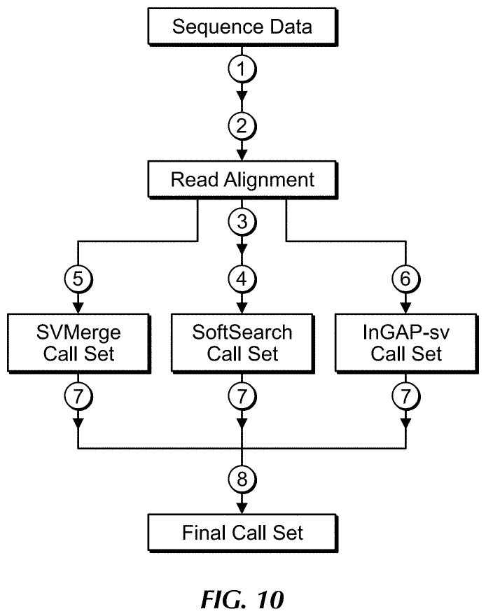

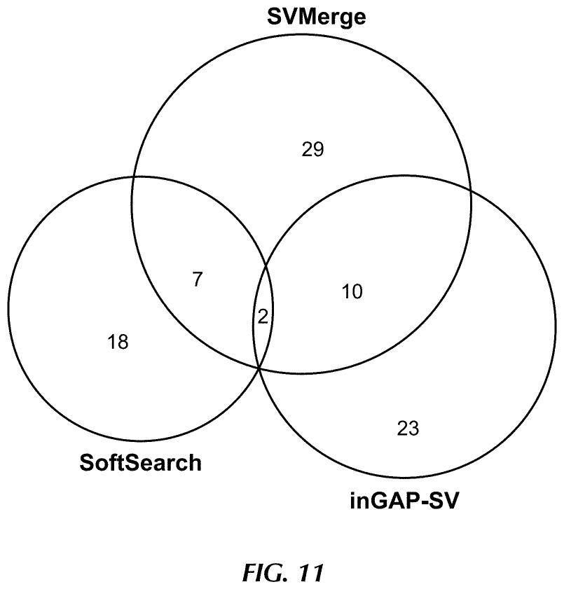

Briefly, SVMerge is a SV-detection platform which implements the RP algorithm BreakDancer (83), RP and SR algorithm Pindel (84), and an algorithm that clusters single-end mapped reads to detect insertions (85). The SVMerge pipeline implements its constituent SV callers, filters and merges the variant calls, then computationally validates breakpoints by Velvet de novo assembly (85). Softsearch is a RP and SR algorithm that is also the only available SV detection platform, which has been experimentally validated for high performance with custom resequencing data (86,87). InGAP-SV is an RD and RP algorithm that uses depth of coverage signatures to identify putative SVs, then refines and categorizes the variants based on RP signals (88). By integrating the output of these three programs, the strengths of all available SV detection algorithms were leveraged and incorporated the best available method for custom resequencing data (FIGS. 10, 11).

Default parameters were used for each SV calling platform, except where a minimum of 25× sequence coverage across all platforms was used to call an SV and a minimum of five reads to form a single-end cluster (Table 17).

TABLE 17
Parameters for in silico annotation of structural variants for A) SVMerge, B) SoftSearch, and C) InGAP-SV.
A.

| | | | Default | Value |
|---|---|---|---|---|
| Parameter Flag | | Parameter Definition | Value | Used |
| BDconfParams | -c | Number of standard deviations away from mean insert | 4 | 4 |
| | | size for read pair mapping to be considered discordant; | | |
| | | Passed to BreakDancer | | |
| | -n | Number of observations required to estimate mean and | 10000 | 10000 |
| | | standard deviation of insert size; Passed to BreakDancer | | |
| Bdparams | -c | Number of standard deviations away from mean insert | 3 | 3 |
| | | size for read pair mapping to be considered discordant; | | |
| | | Passed to BreakDancer | | |
| | -m | Maximum SV size callable; Passed to BreakDancer | 5000000 | 5000000 |
| | -q | Minimum mapping quality used in SV determination; | 25 | 25 |
| | | Passed to BreakDancer | | |
| BD copynum | | Ploidy of Organism; Passed to BreakDancer | 2 | 2 |
| PDoptParams | -x | Maximum SV size callable; Passed to Pindel. Note: 5 | 5 | 5 |
| | | corresponds to 32,368bp | | |
| | -v | Minimum Inversion size callable; Passed to Pindel | 1000 | 1000 |
| SECqual | | Minimum mapping quality used in SV determination; | 20 | 25 |
| | | Passed to SECluster | | |
| SECmin | | Minimum number of reads in either the forward or | 5 | 5 |
| | | reverse cluster, when clusters are paired. | | |
| SECminCluster | | Minimum number of reads to form a single-end | 3 | 5 |
| | | forward or reverse cluster. | | |
| SECmax | | Maximum number of reads allowed in a cluster. | 500 | 500 |
| | BDscore | Score cut-off for data from BreakDancer | 25 | 25 |
| Filtering Step | BDrs | Minimum number of supporting read pairs for data | 2 | 2 |
| | | from BreakDancer | | |
| | PDscore | Score cut-off for data from Pindel | 30 | 30 |
| | PDsupports | Minimum number of supporting read pairs for data | 10 | 10 |
| | | from Pindel | | |
| Hashlen | | Hash-length for assembly; Passed to Velvet | 29 | 29 |
| Library Insert | | Average insert size | NA | See Table S2 |
| Size | | | | |

| | | | |
|---|---|---|---|
| | | Default | Value |
| Parameter | Parameter Definition | Value | Used |

B.

| | | | |
|---|---|---|---|
| q | Minimum mapping quality used in SV determination | 20 | 25 |
| l | Minimum length of soft-clipped segment used in SV determination | 10 | 10 |
| r | Minimum soft-clipped read depth used in SV determination | 10 | 10 |
| m | Minimum number of discordant read pairs to support soft clipped event | 10 | 10 |
| s | Number of standard deviations away from mean insert size for read pair | 4 | 4 |
| | mapping to be considered discordant | | |
| d | Minimum distance between soft-clipped segments and discordant read pairs | 300 | 300 |

C.

| | | | |
|---|---|---|---|
| Min quality | Minimum mapping quality used in SV determination | 10 | 25 |
| Min PE support | Minimum number of discordantly mapped read pairs to support SV | 4 | 4 |
| Min SE support | Minimum number of singly-mapped read pairs to support SV | 4 | 4 |
| Max SV size | Maximum SV size callable | 100000 | 1000000 |
| X of std dev | Number of standard deviations away from mean insert size for read pair | 3 | 3 |
| | mapping to be considered discordant | | |

As gaps in highly repetitive regions of the reference genome represent the primary source of false positives in SV discovery (89,90), SV calls from all platforms were filtered with a custom script that removed all variant calls with breakpoints that fell inside gaps, microsatellites, and tandem repeats in the reference genome annotated by the UCSC Table Browser (91). The filtered sets of SV output by each program were merged into a final table and then clustered into a single event if both breakpoints fell within 200 base pairs of each other (92) (FIG. 5). The SV detection platforms used in the pipeline predict the presence or absence of SVs, but not whether an animal is homozygous or heterozygous for a given SV. It is more biologically plausible that a given SV is heterozygous due to unequal crossing over that mediates structural variation in the WBSCR17 in humans that result in hemizygous changes (20), and that large homozygous deletions are often fatal (50). Thus, SV-positive loci were coded as heterozygous. Genotypes were assigned with a custom script (Table 18).

TABLE 18
Structural variant genotype per individual.

| Animal ID: | 2769 | 2771 | 2772 | 2773 | 2774 | 2775 | 2776 | 2777 | 2778 | 2789 | 2780 | 2781 |
|---|---|---|---|---|---|---|---|---|---|---|---|---|
| Cfa.1 | 0 | 0 | 0 | 0 | 1 | 0 | 0 | 0 | 0 | 0 | 0 | 0 |
| Cfa.2 | 0 | 0 | 0 | 0 | 0 | 0 | 0 | 0 | 0 | 0 | 0 | 0 |
| Cfa.3 | 0 | 0 | 0 | 1 | 1 | 0 | 0 | 0 | 0 | 0 | 0 | 1 |
| Cfa.4 | 0 | 0 | 0 | 0 | 1 | 0 | 0 | 0 | 0 | 0 | 0 | 0 |
| Cfa.5 | 0 | 0 | 0 | 0 | 0 | 0 | 0 | 0 | 0 | 0 | 1 | 0 |
| Cfa.6 | 0 | 0 | 0 | 0 | 0 | 0 | 0 | 0 | 0 | 0 | 0 | 0 |
| Cfa.7 | 0 | 0 | 0 | 0 | 0 | 0 | 0 | 0 | 0 | 0 | 0 | 0 |
| Cfa.8 | 0 | 0 | 0 | 0 | 0 | 0 | 0 | 0 | 0 | 0 | 0 | 0 |
| Cfa.9 | 0 | 0 | 0 | 0 | 0 | 1 | 0 | 0 | 0 | 0 | 0 | 0 |
| Cfa.10 | 0 | 0 | 0 | 0 | 0 | 0 | 0 | 0 | 0 | 0 | 0 | 0 |
| Cfa.11 | 0 | 0 | 0 | 0 | 0 | 0 | 0 | 0 | 0 | 0 | 0 | 0 |
| Cfa.12 | 0 | 0 | 0 | 0 | 0 | 0 | 0 | 0 | 0 | 0 | 0 | 0 |
| Cfa.13 | 0 | 0 | 0 | 0 | 0 | 0 | 0 | 0 | 0 | 0 | 0 | 0 |
| Cfa.14 | 0 | 0 | 0 | 0 | 0 | 0 | 0 | 0 | 0 | 0 | 0 | 0 |
| Cfa.15 | 0 | 0 | 0 | 0 | 0 | 0 | 0 | 0 | 0 | 0 | 0 | 0 |
| Cfa.16 | 0 | 0 | 0 | 0 | 0 | 0 | 0 | 0 | 0 | 0 | 0 | 1 |
| Cfa.17 | 1 | 0 | 0 | 0 | 0 | 0 | 0 | 1 | 0 | 0 | 0 | 0 |
| Cfa.18 | 0 | 0 | 0 | 0 | 0 | 0 | 0 | 0 | 0 | 0 | 0 | 0 |
| Cfa.19 | 0 | 0 | 0 | 0 | 0 | 0 | 1 | 0 | 0 | 0 | 0 | 0 |
| Cfa.20 | 1 | 0 | 0 | 0 | 0 | 1 | 0 | 1 | 1 | 0 | 0 | 0 |
| Cfa.21 | 0 | 0 | 0 | 0 | 0 | 0 | 0 | 0 | 0 | 0 | 0 | 1 |
| Cfa.22 | 0 | 0 | 1 | 0 | 0 | 0 | 0 | 0 | 1 | 0 | 0 | 1 |
| Cfa.23 | 0 | 0 | 1 | 0 | 0 | 0 | 0 | 0 | 0 | 0 | 0 | 0 |
| Cfa.24 | 1 | 0 | 0 | 0 | 0 | 0 | 0 | 1 | 1 | 0 | 0 | 1 |
| Cfa.25 | 0 | 1 | 0 | 1 | 0 | 1 | 1 | 0 | 1 | 1 | 0 | 1 |
| Cfa.26 | 1 | 1 | 1 | 1 | 0 | 1 | 1 | 1 | 1 | 1 | 1 | 1 |
| Cfa.27 | 0 | 0 | 0 | 0 | 0 | 0 | 0 | 0 | 0 | 0 | 0 | 0 |
| Cfa.28 | 0 | 0 | 0 | 1 | 1 | 1 | 0 | 0 | 1 | 0 | 0 | 1 |
| Cfa.29 | 0 | 0 | 1 | 1 | 1 | 0 | 0 | 1 | 1 | 1 | 0 | 1 |
| Cfa.30 | 0 | 0 | 0 | 0 | 1 | 0 | 0 | 0 | 1 | 0 | 0 | 0 |
| Cfa.31 | 1 | 0 | 0 | 0 | 0 | 0 | 0 | 0 | 0 | 0 | 0 | 0 |
| Cfa.32 | 0 | 1 | 1 | 1 | 1 | 1 | 1 | 1 | 1 | 1 | 1 | 1 |
| Cfa.33 | 1 | 0 | 0 | 0 | 0 | 0 | 0 | 0 | 0 | 0 | 0 | 0 |
| Cfa.34 | 0 | 0 | 0 | 0 | 0 | 0 | 0 | 0 | 0 | 0 | 0 | 0 |
| Cfa.35 | 0 | 0 | 0 | 1 | 0 | 0 | 1 | 1 | 0 | 0 | 0 | 0 |
| Cfa.36 | 1 | 0 | 0 | 0 | 0 | 0 | 0 | 0 | 0 | 0 | 0 | 0 |
| Cfa.37 | 0 | 0 | 0 | 0 | 0 | 0 | 0 | 0 | 0 | 0 | 0 | 0 |
| Cfa.38 | 1 | 0 | 0 | 0 | 0 | 0 | 0 | 0 | 0 | 0 | 0 | 0 |
| Cfa.39 | 0 | 0 | 0 | 0 | 0 | 1 | 0 | 0 | 0 | 0 | 0 | 0 |
| Cfa.40 | 0 | 1 | 0 | 1 | 0 | 0 | 0 | 0 | 0 | 0 | 0 | 0 |
| Cfa.41 | 0 | 0 | 0 | 0 | 0 | 0 | 0 | 0 | 0 | 0 | 0 | 0 |
| Cfa.42 | 0 | 0 | 0 | 0 | 1 | 0 | 1 | 0 | 0 | 0 | 0 | 0 |
| Cfa.43 | 0 | 0 | 0 | 1 | 0 | 0 | 0 | 0 | 0 | 0 | 0 | 0 |
| Cfa.44 | 1 | 1 | 0 | 1 | 1 | 1 | 0 | 1 | 1 | 1 | 1 | 1 |
| Cfa.45 | 1 | 0 | 0 | 0 | 0 | 0 | 0 | 0 | 0 | 0 | 0 | 0 |
| Cfa.46 | 0 | 0 | 0 | 0 | 0 | 0 | 0 | 0 | 0 | 0 | 0 | 0 |
| Cfa.47 | 0 | 1 | 0 | 0 | 0 | 0 | 1 | 0 | 0 | 1 | 0 | 0 |
| Cfa.48 | 1 | 0 | 0 | 0 | 0 | 0 | 0 | 0 | 0 | 0 | 0 | 0 |
| Cfa.49 | 1 | 0 | 0 | 0 | 0 | 0 | 0 | 0 | 0 | 0 | 0 | 0 |
| Cfa.50 | 0 | 0 | 0 | 1 | 1 | 1 | 0 | 1 | 0 | 0 | 0 | 1 |
| Cfa.51 | 0 | 0 | 0 | 0 | 0 | 0 | 0 | 0 | 0 | 0 | 0 | 0 |
| Cfa.52 | 0 | 0 | 0 | 0 | 0 | 0 | 0 | 0 | 0 | 0 | 0 | 0 |
| Cfa.53 | 1 | 0 | 0 | 0 | 0 | 0 | 0 | 0 | 0 | 0 | 0 | 0 |
| Cfa.54 | 0 | 1 | 0 | 0 | 1 | 0 | 0 | 1 | 0 | 0 | 0 | 1 |
| Cfa.55 | 0 | 0 | 0 | 0 | 0 | 0 | 0 | 0 | 0 | 0 | 0 | 0 |
| Cfa.56 | 1 | 0 | 0 | 0 | 0 | 0 | 1 | 0 | 1 | 0 | 0 | 0 |
| Cfa.57 | 0 | 0 | 0 | 0 | 0 | 0 | 0 | 0 | 0 | 0 | 0 | 0 |
| Cfa.58 | 0 | 0 | 0 | 0 | 0 | 0 | 0 | 0 | 0 | 0 | 0 | 0 |
| Cfa.59 | 1 | 1 | 1 | 1 | 1 | 1 | 1 | 1 | 1 | 0 | 1 | 1 |
| Cfa.60 | 0 | 0 | 1 | 0 | 0 | 0 | 1 | 1 | 1 | 1 | 0 | 1 |
| Cfa.61 | 1 | 0 | 0 | 0 | 0 | 0 | 0 | 0 | 0 | 0 | 0 | 0 |
| Cfa.62 | 0 | 0 | 0 | 0 | 1 | 0 | 0 | 0 | 0 | 0 | 0 | 0 |
| Cfa.63 | 0 | 0 | 0 | 0 | 0 | 0 | 0 | 0 | 0 | 0 | 0 | 0 |
| Cfa.64 | 0 | 0 | 0 | 1 | 0 | 0 | 0 | 0 | 0 | 0 | 0 | 0 |
| Cfa.65 | 0 | 0 | 0 | 0 | 0 | 0 | 0 | 0 | 0 | 0 | 0 | 0 |
| Cfa.66 | 1 | 1 | 1 | 1 | 0 | 1 | 1 | 1 | 1 | 0 | 1 | 1 |
| Cfa.67 | 0 | 0 | 0 | 0 | 0 | 0 | 0 | 1 | 0 | 0 | 0 | 0 |
| Cfa.68 | 0 | 0 | 0 | 0 | 1 | 0 | 1 | 0 | 1 | 1 | 0 | 1 |
| Cfa.69 | 1 | 0 | 0 | 0 | 0 | 1 | 0 | 0 | 0 | 1 | 0 | 1 |
| Cfa.70 | 0 | 0 | 0 | 0 | 0 | 0 | 0 | 0 | 0 | 0 | 0 | 0 |
| Cfa.71 | 0 | 0 | 0 | 0 | 0 | 0 | 0 | 0 | 0 | 0 | 0 | 0 |
| Cfa.72 | 0 | 0 | 0 | 0 | 0 | 0 | 0 | 0 | 0 | 1 | 0 | 1 |
| Cfa.73 | 0 | 0 | 0 | 0 | 0 | 0 | 0 | 0 | 0 | 0 | 0 | 1 |
| Cfa.74 | 0 | 0 | 0 | 0 | 1 | 0 | 0 | 0 | 0 | 0 | 0 | 0 |
| Cfa.75 | 1 | 0 | 0 | 0 | 0 | 0 | 0 | 0 | 0 | 0 | 0 | 0 |
| Cfa.76 | 0 | 0 | 0 | 0 | 0 | 0 | 0 | 0 | 0 | 0 | 0 | 0 |
| Cfa.77 | 1 | 0 | 0 | 0 | 0 | 0 | 0 | 0 | 0 | 1 | 0 | 1 |
| Cfa.78 | 0 | 0 | 0 | 0 | 0 | 0 | 0 | 0 | 1 | 0 | 1 | 1 |
| Cfa.79 | 0 | 0 | 1 | 1 | 1 | 0 | 0 | 0 | 0 | 0 | 0 | 0 |
| Cfa.80 | 0 | 0 | 0 | 0 | 0 | 0 | 0 | 0 | 0 | 0 | 0 | 0 |
| Cfa.81 | 0 | 0 | 0 | 0 | 0 | 0 | 0 | 0 | 0 | 0 | 0 | 0 |
| Cfa.82 | 0 | 0 | 0 | 0 | 0 | 0 | 0 | 0 | 0 | 0 | 0 | 0 |
| Cfa.83 | 0 | 0 | 0 | 1 | 1 | 1 | 0 | 0 | 0 | 0 | 0 | 0 |
| Cfa.84 | 0 | 0 | 0 | 0 | 0 | 1 | 0 | 0 | 0 | 0 | 0 | 0 |
| Cfa.85 | 0 | 0 | 0 | 0 | 0 | 0 | 0 | 0 | 0 | 0 | 0 | 0 |
| Cfa.86 | 0 | 0 | 0 | 0 | 1 | 0 | 1 | 0 | 0 | 1 | 0 | 0 |
| Cfa.87 | 0 | 0 | 0 | 0 | 0 | 0 | 0 | 0 | 0 | 0 | 0 | 0 |
| Cfa.88 | 0 | 1 | 0 | 0 | 0 | 0 | 0 | 0 | 0 | 0 | 0 | 0 |
| Cfa.89 | 0 | 0 | 0 | 0 | 0 | 1 | 0 | 0 | 0 | 0 | 0 | 0 |
| Animal ID: | 2782 | 2783 | 2784 | 2785 | 2786 | 2787 | 2788 | 2789 | 2790 | 2791 | 2792 | 2793 |
| Cfa.1 | 0 | 0 | 0 | 0 | 0 | 0 | 0 | 0 | 0 | 0 | 0 | 0 |
| Cfa.2 | 0 | 0 | 0 | 0 | 0 | 0 | 0 | 0 | 0 | 0 | 1 | 0 |
| Cfa.3 | 0 | 0 | 0 | 1 | 1 | 0 | 0 | 0 | 0 | 0 | 1 | 1 |
| Cfa.4 | 0 | 0 | 0 | 0 | 0 | 0 | 0 | 0 | 0 | 0 | 0 | 0 |
| Cfa.5 | 0 | 0 | 0 | 0 | 0 | 0 | 0 | 0 | 0 | 0 | 0 | 0 |
| Cfa.6 | 0 | 0 | 0 | 0 | 1 | 1 | 1 | 1 | 0 | 1 | 1 | 1 |
| Cfa.7 | 0 | 0 | 1 | 0 | 1 | 0 | 1 | 0 | 0 | 0 | 0 | 1 |
| Cfa.8 | 0 | 0 | 0 | 0 | 1 | 1 | 1 | 1 | 0 | 1 | 1 | 1 |
| Cfa.9 | 0 | 0 | 0 | 0 | 1 | 0 | 0 | 1 | 1 | 1 | 0 | 1 |
| Cfa.10 | 0 | 0 | 0 | 0 | 0 | 0 | 0 | 0 | 0 | 1 | 0 | 0 |
| Cfa.11 | 0 | 0 | 0 | 0 | 0 | 0 | 0 | 0 | 1 | 0 | 0 | 0 |
| Cfa.12 | 0 | 0 | 0 | 0 | 0 | 1 | 0 | 0 | 0 | 0 | 0 | 0 |
| Cfa.13 | 0 | 0 | 0 | 0 | 0 | 1 | 0 | 0 | 0 | 0 | 0 | 0 |
| Cfa.14 | 0 | 0 | 0 | 0 | 0 | 0 | 0 | 0 | 0 | 0 | 1 | 0 |
| Cfa.15 | 0 | 0 | 0 | 0 | 0 | 0 | 0 | 0 | 0 | 1 | 0 | 0 |
| Cfa.16 | 0 | 0 | 0 | 0 | 0 | 0 | 0 | 0 | 0 | 0 | 0 | 0 |
| Cfa.17 | 1 | 0 | 1 | 0 | 0 | 0 | 0 | 0 | 0 | 0 | 0 | 0 |
| Cfa.18 | 0 | 0 | 0 | 1 | 0 | 0 | 0 | 0 | 0 | 0 | 0 | 0 |
| Cfa.19 | 0 | 0 | 0 | 0 | 0 | 0 | 0 | 0 | 0 | 0 | 0 | 0 |
| Cfa.20 | 1 | 0 | 0 | 0 | 0 | 0 | 0 | 1 | 1 | 0 | 0 | 0 |
| Cfa.21 | 0 | 0 | 0 | 0 | 0 | 0 | 0 | 0 | 0 | 0 | 0 | 0 |
| Cfa.22 | 0 | 0 | 0 | 1 | 0 | 0 | 1 | 0 | 0 | 0 | 0 | 0 |
| Cfa.23 | 0 | 0 | 0 | 0 | 0 | 0 | 0 | 0 | 0 | 0 | 0 | 0 |
| Cfa.24 | 1 | 0 | 0 | 0 | 0 | 0 | 0 | 0 | 0 | 0 | 0 | 0 |
| Cfa.25 | 0 | 1 | 1 | 0 | 1 | 1 | 0 | 1 | 1 | 1 | 1 | 1 |
| Cfa.26 | 1 | 1 | 1 | 0 | 0 | 0 | 0 | 1 | 1 | 1 | 1 | 1 |
| Cfa.27 | 0 | 0 | 0 | 0 | 0 | 0 | 0 | 1 | 1 | 1 | 0 | 0 |
| Cfa.28 | 0 | 0 | 0 | 0 | 0 | 0 | 0 | 0 | 0 | 0 | 0 | 1 |
| Cfa.29 | 0 | 0 | 1 | 0 | 0 | 1 | 0 | 1 | 1 | 0 | 0 | 0 |
| Cfa.30 | 0 | 0 | 0 | 0 | 0 | 0 | 0 | 0 | 0 | 0 | 0 | 0 |
| Cfa.31 | 1 | 0 | 0 | 0 | 0 | 0 | 0 | 0 | 0 | 0 | 0 | 0 |
| Cfa.32 | 0 | 1 | 0 | 1 | 0 | 1 | 0 | 1 | 1 | 1 | 1 | 1 |
| Cfa.33 | 1 | 0 | 0 | 0 | 0 | 0 | 0 | 0 | 0 | 0 | 0 | 0 |
| Cfa.34 | 0 | 0 | 0 | 0 | 0 | 1 | 0 | 0 | 0 | 0 | 0 | 0 |
| Cfa.35 | 0 | 0 | 0 | 1 | 0 | 1 | 0 | 1 | 1 | 1 | 0 | 1 |
| Cfa.36 | 1 | 0 | 0 | 0 | 0 | 0 | 0 | 0 | 0 | 0 | 0 | 0 |
| Cfa.37 | 0 | 0 | 0 | 0 | 0 | 0 | 0 | 0 | 0 | 0 | 0 | 1 |
| Cfa.38 | 1 | 0 | 0 | 0 | 0 | 0 | 0 | 0 | 0 | 0 | 0 | 0 |
| Cfa.39 | 0 | 0 | 0 | 0 | 0 | 0 | 0 | 0 | 0 | 0 | 0 | 1 |
| Cfa.40 | 0 | 1 | 1 | 1 | 0 | 0 | 0 | 1 | 0 | 1 | 1 | 1 |
| Cfa.41 | 0 | 0 | 0 | 0 | 0 | 0 | 0 | 1 | 1 | 0 | 0 | 0 |
| Cfa.42 | 0 | 0 | 0 | 0 | 0 | 0 | 0 | 1 | 0 | 1 | 1 | 1 |
| Cfa.43 | 0 | 0 | 0 | 0 | 0 | 0 | 0 | 0 | 0 | 0 | 0 | 0 |
| Cfa.44 | 1 | 1 | 1 | 1 | 1 | 1 | 1 | 1 | 1 | 1 | 1 | 1 |
| Cfa.45 | 1 | 0 | 0 | 0 | 0 | 0 | 0 | 0 | 0 | 0 | 0 | 0 |
| Cfa.46 | 0 | 0 | 0 | 0 | 0 | 1 | 0 | 0 | 0 | 0 | 0 | 0 |
| Cfa.47 | 0 | 1 | 0 | 0 | 1 | 0 | 1 | 1 | 1 | 1 | 1 | 1 |
| Cfa.48 | 1 | 0 | 0 | 0 | 0 | 0 | 0 | 0 | 0 | 0 | 0 | 0 |
| Cfa.49 | 1 | 0 | 0 | 0 | 0 | 0 | 0 | 0 | 0 | 0 | 0 | 0 |
| Cfa.50 | 0 | 0 | 1 | 0 | 1 | 1 | 0 | 1 | 1 | 1 | 1 | 1 |
| Cfa.51 | 0 | 0 | 0 | 1 | 0 | 0 | 0 | 0 | 0 | 0 | 0 | 0 |
| Cfa.52 | 0 | 0 | 0 | 0 | 0 | 0 | 0 | 0 | 1 | 0 | 0 | 0 |
| Cfa.53 | 1 | 0 | 0 | 0 | 0 | 0 | 0 | 0 | 0 | 0 | 0 | 0 |
| Cfa.54 | 0 | 1 | 1 | 1 | 0 | 1 | 1 | 1 | 0 | 0 | 1 | 1 |
| Cfa.55 | 0 | 0 | 0 | 0 | 0 | 0 | 0 | 0 | 1 | 0 | 0 | 1 |
| Cfa.56 | 1 | 0 | 0 | 1 | 1 | 1 | 0 | 1 | 0 | 0 | 0 | 1 |
| Cfa.57 | 0 | 0 | 0 | 0 | 1 | 1 | 0 | 1 | 1 | 1 | 1 | 0 |
| Cfa.58 | 0 | 0 | 0 | 0 | 1 | 0 | 0 | 0 | 0 | 0 | 0 | 0 |
| Cfa.59 | 1 | 1 | 1 | 1 | 1 | 1 | 0 | 1 | 1 | 1 | 1 | 1 |
| Cfa.60 | 0 | 0 | 0 | 1 | 0 | 0 | 0 | 1 | 0 | 0 | 1 | 1 |
| Cfa.61 | 1 | 0 | 0 | 0 | 0 | 0 | 0 | 0 | 0 | 0 | 0 | 0 |
| Cfa.62 | 0 | 0 | 0 | 0 | 0 | 0 | 0 | 0 | 0 | 0 | 0 | 0 |
| Cfa.63 | 0 | 0 | 0 | 0 | 0 | 0 | 0 | 0 | 1 | 0 | 0 | 0 |
| Cfa.64 | 0 | 0 | 0 | 0 | 0 | 0 | 0 | 0 | 1 | 0 | 0 | 0 |
| Cfa.65 | 0 | 0 | 0 | 0 | 0 | 0 | 0 | 1 | 0 | 0 | 0 | 0 |
| Cfa.66 | 1 | 0 | 0 | 0 | 0 | 0 | 0 | 0 | 1 | 0 | 0 | 0 |
| Cfa.67 | 0 | 0 | 1 | 0 | 0 | 0 | 0 | 0 | 0 | 0 | 0 | 0 |
| Cfa.68 | 0 | 0 | 0 | 1 | 1 | 1 | 0 | 1 | 1 | 1 | 0 | 0 |
| Cfa.69 | 1 | 0 | 0 | 0 | 0 | 1 | 0 | 1 | 0 | 1 | 1 | 0 |
| Cfa.70 | 0 | 0 | 0 | 0 | 0 | 0 | 0 | 0 | 1 | 0 | 0 | 0 |
| Cfa.71 | 0 | 0 | 0 | 1 | 0 | 0 | 0 | 0 | 0 | 0 | 0 | 0 |
| Cfa.72 | 0 | 0 | 0 | 1 | 0 | 0 | 0 | 1 | 0 | 0 | 0 | 0 |
| Cfa.73 | 0 | 0 | 0 | 0 | 0 | 0 | 0 | 0 | 0 | 0 | 0 | 0 |
| Cfa.74 | 0 | 0 | 0 | 0 | 0 | 0 | 0 | 0 | 0 | 0 | 0 | 0 |
| Cfa.75 | 1 | 0 | 0 | 0 | 0 | 0 | 0 | 0 | 1 | 0 | 1 | 0 |
| Cfa.76 | 0 | 0 | 0 | 0 | 0 | 0 | 0 | 1 | 0 | 0 | 0 | 0 |
| Cfa.77 | 1 | 0 | 0 | 1 | 1 | 1 | 0 | 1 | 1 | 1 | 1 | 0 |
| Cfa.78 | 0 | 0 | 1 | 0 | 0 | 0 | 0 | 1 | 0 | 0 | 1 | 1 |
| Cfa.79 | 0 | 0 | 0 | 1 | 0 | 0 | 0 | 1 | 0 | 0 | 1 | 1 |
| Cfa.80 | 0 | 0 | 0 | 0 | 0 | 0 | 1 | 0 | 0 | 0 | 0 | 0 |
| Cfa.81 | 0 | 0 | 1 | 0 | 0 | 0 | 0 | 0 | 0 | 0 | 0 | 0 |
| Cfa.82 | 0 | 0 | 0 | 0 | 0 | 0 | 0 | 1 | 1 | 0 | 0 | 1 |
| Cfa.83 | 0 | 0 | 0 | 0 | 1 | 0 | 1 | 0 | 1 | 0 | 1 | 0 |
| Cfa.84 | 0 | 0 | 0 | 0 | 0 | 0 | 0 | 0 | 0 | 0 | 0 | 0 |
| Cfa.85 | 0 | 0 | 0 | 0 | 0 | 0 | 0 | 1 | 0 | 0 | 0 | 0 |
| Cfa.86 | 0 | 0 | 0 | 1 | 0 | 0 | 0 | 0 | 1 | 0 | 0 | 0 |
| Cfa.87 | 0 | 0 | 0 | 0 | 1 | 0 | 0 | 0 | 0 | 0 | 0 | 0 |
| Cfa.88 | 0 | 1 | 0 | 0 | 0 | 0 | 0 | 0 | 0 | 0 | 0 | 0 |
| Cfa.89 | 0 | 0 | 0 | 0 | 0 | 0 | 0 | 0 | 0 | 0 | 0 | 0 |

### Example 11—Candidate Region Association Test

The univariate linear mixed model implemented in the program GEMMA (93) was used to test for associations between SVs and each of the three behavioral indices. GEMMA's univariate module fits a set of genotypes and corresponding phenotypes to fit a univariate linear mixed model that accounts for fixed effects, population stratification, and sample structure. For each variant, the univariate model tests the alternative hypothesis H₁: β≠0 against the null hypothesis H₀: β=0, using the Wald, likelihood ratio, and score test statistics, where β is the effect size of each variant on the phenotype of interest. Population stratification is accounted for using either a centered or standardized relatedness matrix as a random effect, where the authors recommend a centered matrix for non-human organisms. Three univariate models were thus implemented: the first estimating associations between SVs and attentional bias to social stimuli (ABS model), the second between SVs and hyper-sociability (HYP model), and the third between SVs and social interest in strangers (SIS model). For each univariate model, the centered relatedness matrix was estimated from SNP genotypes in the target region by GEMMA, and incorporated to account for relatedness and population structure among the samples. SNP genotypes were used in calculating the relatedness matrix in place of SV genotypes, as there was more than an order of magnitude more SNP genotypes than SV genotypes (4844 vs. 89) on which to base the estimation. Negative values in the relatedness matrix, indicating that there was less relatedness between a given pair of individuals than would be expected between two randomly chosen individuals, were set to 0 in the resulting matrix (94,95). Sex and age were used as covariates. Only SV with minor allele frequency (MAF)>0.025 were tested (96). The Bonferroni correction for multiple comparisons was used in conjunction with the simpleM method for accounting for linkage disequilibrium among variants (97) to establish significance thresholds. With simpleM (http://simplem.sourceforge.net/), the effective number of independent tests were estimated as Meff=21, corresponding to a significance threshold of p=2.38×10⁻³ (Bonferroni cutoff of α=0.05 for 21 independently tested SVs). The likelihood ratio test was used to determine p-values. Because the ABS phenotype was calculated as a proportion, the arcsin transformation was applied before all analyses; all other phenotypes were not transformed.

GEMMA's multivariate linear mixed models estimate the association between a given variant and all phenotypes of interest simultaneously, accounting for the correlation between the phenotypes and generally exhibiting greater statistical power than univariate linear mixed models. Specifically, GEMMA's multivariate module fits a set of genotypes and corresponding phenotypes to a multivariate linear mixed model that accounts for fixed effects, population stratification and sample structure. For each variant, GEMMA tests the alternative hypothesis H₁: β≠0 against the null hypothesis H₀: β=0, using the Wald, likelihood ratio, and score test statistics, where 3 is the effect size of each variant for all phenotypes. In addition to the univariate models implemented for each phenotype individually, GEMMA's multivariate linear mixed model was used to estimate associations between SVs and several behavioral phenotypes simultaneously. Two multivariate models were implemented with the same model parameters and data transformation used in the univariate models: one estimating associations between SVs and the indices of human-directed sociability (Behavioral Index model) and the other estimating associations between SVs and the first three PCs of social behavior (PC model).

To investigate the possibility that SVs are associated with species membership (dog versus wolf), an association scan of each SV locus with species membership was conducted with PLINK (98) (Table 11). Variants strongly associated with social behavior, but not species membership, are particularly robust candidates for mediators of social behavior.

### Example 12—PCR Validation and Analysis of Structural Variants

An attempt was made to design primers flanking all SVs significantly associated with human-directed social behavior (Table 9) as well as two other SVs that were suggestive of an association but did not pass the significance threshold (univariate model: HYP and Cfa6.6, β±se=−138.8±33.62, p=5.75×10⁻³; ABS and Cfa6.83, β±se=−0.0640.09, p=6.90×10⁻³). Primers were designed based on the dog reference genome (Canfam3.1) with Primer3 (99) (Table 19).

TABLE 19
Primer sequences used for PCR and gel-based validation
of structural variants.

| | | Amplicon size (bp): | |
|---|---|---|---|
| Locus | Primer sequence | Insertion | Wildtype |
| Cfa6.6 | Forward: CCCCTTCAGCCAGCATATAA | 555 | 357 |
| | Reverse: TTCTCTGGGCTGTCTGGACT | | |
| Cfa6.6 | Forward: AAGTTTCTCTGATGGAAAACACA | 278 | 90 |
| (internal) | Reverse: GGTGGCTGGAAATTTCAGTAG | | |
| Cfa6.7 | Forward: TGGAGCCATGATTAGGAAGG | 504 | 269 |
| | Reverse: TAAGGAAGGACCCCATTTCC | | |
| Cfa6.66 | Forward: TGCTGCTTCATGTTCTGTGA | 505 | 215 |
| | Reverse: TGGTGCATTAGCTTTGGTTG | | |
| Cfa6.83 | Forward: AACCACAGGAACAAAACCTCA | 400 | 184 |
| | Reverse: CCTCCTGTTGGACATTTGGA | | |

Primers that amplified Cfa6.3 and Cfa6.72 could not be designed, and thus high-confidence codominant genotypes could only be obtained for Cfa6.6, Cfa6.7, Cfa6.66, and Cfa6.83. Cfa6.3 is ˜40 bp downstream of a 300 bp gap in the reference genome. It is possible that this gap caused a false positive during the in silico annotation of this locus, as any sequencing into the gap would not map to the reference and could instead be interpreted as an insertion by SV annotation algorithms.

For the 24 dogs and wolves in the targeted resequencing study, along with a broader sampling of wild canids and dog breeds, each SV locus was PCR amplified and genotypes were called based on banding patterns in agarose gel electrophoresis (FIG. 8). PCR conditions were as follows: 0.2 mM dNTPs, 2.5 mM MgCl₂, 0.1 mg/mL bovine serum albumin, 0.2 uM each primer, 0.75 units Amplitaq Gold (Thermo Fisher Scientific), 1× Gold buffer, and ˜10 ng genomic DNA. Cycling conditions were: 10 m at 95° C., followed by 30 cycles of 30 s at 95° C., 30 s at 60° C. (30 s at 55° C. for Cfa6.83), and 45 s at 72° C., and a final 10 m extension at 72° C. Ten to 15 uL of PCR product were run on a 1.8% agarose gel and imaged for genotype calling (FIG. 6). To confirm that the SVs consist of transposable elements (TEs), PCR products for three individuals per homozygous genotype were Sanger sequenced, and assembled and aligned to CanFam3.1 in Geneious. Low-complexity regions in the TE at all three loci resulted in poor sequence quality, and locus Cfa6.6 required additional internal primers to sequence across the TE. Alignment to the dog reference genome shows that SV lengths are very similar to the in silico estimates, and that in each case the TEs are fully contained within the SV. Cfa6.6 is 196 bp (includes 188 bp TE); Cfa6.7 is 229 bp (193 bp TE); Cfa6.66 is 259 bp (187 bp TE), and Cfa6.83 is 216 bp (182 bp TE).

PCR was used to amplify and electrophoresis methods to genotype four SVs in a panel of wild canids (n, gray wolves: Europe=12, India/Iran=7, China=3, Middle East=14, North America=15; coyotes n=13), the 16 domestic dogs from the initial sequencing efforts, and 201 domestic dogs from 13 AKC registered breeds (n, dogs: Alaskan Malamute=13, Bernese Mountain dog=20, Border Collie=20, Boxer=13, Basenji=7, Cairn Terrier=18, Golden Retriever=16, Great Pyrenees=17, Jack Russell Terrier=17, Miniature Poodle=10, Miniature Schnauzer=16, Pug=19, Saluki=15). 17 semi-domestic populations were also genotyped, representing New Guinea Singing dogs (NGSD, n=3), Pariah dogs from Saudi Arabia (n=4), and village dogs from two locations (Africa, n=5; Puerto Rico, n=5). Though an ideal design would include a large sampling of individuals from an experimental dog-wolf cross (e.g. F1 hybrids and backcrosses), this is not possible to construct in the United States as it would require generating an animal colony with years of selected breeding. An alternative method would be to explore genome editing with CRISPR/Cas9, which has only recently been shown to work in canines (100).

Breeds from across multiple breed-type clades were selected, representing different ancestries and behavioral functions. Each breed was phenotyped according to AKC behavioral stereotypes (41) into a category of either seeking or avoiding attention (Seeks attention: Bernese Mountain dog, Border collie, Boxer, Golden retriever, Jack Russell terrier, Miniature poodle, Pug; Avoids attention: Alaskan malamute, Basenji, Cairn terrier, Great Pyrenees, Miniature schnauzer, Saluki, and all semi-domestic dogs). The breeds that were classified as “seeks attention” were those that typically attempted to engage with humans, familiar or unfamiliar (41). It was not required that these breeds be gregarious or hyper-social, in that they actively seek any human attention; rather, that they show preference for working with humans, spending time, receiving affection, or offering behaviors to human counterparts. Conversely, the breeds that “avoid attention” are those that would classically be categorized as “aloof” or “independent”. They were either bred to exist on the periphery of human life, or tend to opt for individual pursuits.

### Example 13—Genome-wide SNP survey

Genome-wide SNP genotypes were collected using the Affymetrix Axiom K9HDSNPA (643,641 loci) and Axiom K9HDSNPB (625,577 loci) arrays with an average concentration of 26.5 ng/uL for 11 of the 24 individuals with behavioral phenotypes (n_{dog}=5; n_{wolf}=6). Samples with a dish QC value≥0.82 and call rate≥97% were retained. SNP genotype quality control and processing identified that 794,665 SNPs, 56.3% of K9HDSNPA (250,545 loci) and 87% of K9HDSNPB (544,120 loci), passed filtering metrics. Affymetrix recommended a subset of 544,120 loci (referred to as 544K SNPs) to be included for all downstream analyses. PLINK was used to obtain a pruned set of 25,510 uncorrelated and unlinked SNPs with the argument—indep-pairwise 50 5 0.2, then conducted a PCA with the program flashPCA (101) (FIG. 9). A binary association test in PLINK was also conducted on the binary phenotype of species membership. Further, a quantitative association test was conducted using the quantitative behavioral traits and a significance threshold of p<0.005, testing each of the behaviors (ABS, HYP, SIS) independently, then jointly. Similar to the regression of the targeted resequencing data described above, a univariate regression analysis was completed with GEMMA on the 544K SNP set and the quantitative behavioral phenotypes of ABS, HYP, and SIS. Kinship information was incorporated via a relatedness matrix. The likelihood ratio test significance threshold was adjusted to p<1^{st} percentile to identify candidate regions. Gene ontology (GO) enrichment analysis was conducted in WebGestalt (102,103) using the reference genome as the reference set of genes, the hypergeometric test for evaluating the level of term enrichment and adjusted the significance threshold due to multiple testing using the Benjamini & Hochberg method (104). A term was considered significant if the adjusted value wasp<0.05.

### Example 14—Ethics

All subjects were volunteered by their owners/caretakers and remained in their care throughout the study. Experimental procedures were evaluated and approved by Oregon State University IACUC, protocol #4444. Laboratory methods were conducted under the approved IACUC protocol #2008A-14 of Princeton University. Institutional IACUC guidelines were followed with animal subjects.

### REFERENCES

1. Frank, H., Evolution of canine information processing under conditions of natural and artificial selection. Zeitschrift fûr Tierpsychologie 53, 389-399 (1980).
2. Miklósi, Á., Topal, J., Csányi, V., Comparative social cognition: what can dogs teach us?Anim. Behav. 67, 995-1004 (2004).
3. Hare, B., Tomasello, M., Human-like social skills in dogs? Trends. Cogn. Sci. 9, 439-444 (2005).
4. Udell, M. A., Dorey, N. R., Wynne, C. D., What did domestication do to dogs? A new account of dogs' sensitivity to human actions. Biol. Rev. 85, 327-345 (2010).
5. Trut, L., Oskina, I., Kharlamova, A., Animal evolution during domestication: the domesticated fox as a model. Bioessays 31(3), 349-360 (2009).
6. Nagasawa, M., Mitsui, S., En, S., Ohtani, N., Ohta, M., Sakuma, Y., Onaka, T., Mogi, K., Kikusui, T., Oxytocin-gaze positive loop and the coevolution of human-dog bonds. Science 348, 333-336 (2015).
7. Bentosela, M., Wynne, C. D., D'Orazio, M., Elgier, A., Udell, M. A. R., Sociability and gazing toward humans in dogs and wolves: Simple behaviors with broad implications. J. Exp. Anal. Behav. 105, 68-75 (2016).
8. Udell, M. A., When dogs look back: inhibition of independent problem-solving behaviour in domestic dogs (Canis lupus familiaris) compared with wolves (Canis lupus). Biol. Letters 11, 20150489 (2015).
9. Jones, P., Chase, K., Martin, A., Davern, P., Ostrander, E. A., Lark, K. G., Single-nucleotide-polymorphism-based association mapping of dog stereotypes. Genetics 179(2), 1033-1044 (2008).
10. Parker, H. G., Kim, L. V., Sutter, N. B. Carlson, S., Lorentzen, T. D., Malek, T. B., Johnson, G. S., DeFrance, H. B., Ostrander, E. A., Kruglyak, L., Genetic structure of the purebred domestic dog. Science 304, 1160-1164 (2004).
11. Serpell, J. A., Hsu, Y., Effects of breed, sex, and neuter status on trainability in dogs. Anthrozoös 18, 196-207 (2005). [symbol]
12. Svartberg, K., Breed-typical behavior in dogs—historical remnants or recent constructs?Appl. Anim. Behav. Sci. 96, 293-313 (2006). [symbol]
13. Duffy, D. L., Hsu, Y., Serpell, J. A., Breed differences in canine aggression. Appl. Anim. Behav. Sci. 114, 441-460 (2008).
14. Ley, J. M., Bennett, P. M., Coleman, G. J., A refinement and validation of the Monash Canine Personality Questionnaire (MCPQ). Appl. Anim. Behav. Sci. 116, 220-227 (2009).
15. Turcsán, B., Kubinyi, E., Miklósi, A., Trainability and boldness traits differ between dog breed clusters based on conventional breed categories and genetic relatedness. Appl. Anim. Behav. Sci. 132, 61-70 (2011).
16. Vaysse, A., Ratnakumar, A., Derrien, T., Axelsson, E., Rosengren Pielberg, G., Sigurdsson, S., Fall, T., Seppälä, E. H., Hansen, M. S. T., Lawley, C. T., Karlsson, E. K., The LUPA Consortium, Bannasch, D., Vilà, C., Lohi, H., Galibert, F., Fredholm, M., Häggström, J., Hedhammar, A., André, C., Lindblad-Toh, K., Hitte, C., Webster, M. T., Identification of genomic regions associated with phenotypic variation between dog breeds using selection mapping. PLoS Genet. 7(10), e1002316 (2011).
17. Serpell, J. A., Duffy, D. L., Dog breeds and their behavior. A. Horowitz (ed.), Domestic Dog Cognition and Behavior, Springer p 31-57 (2014).
18. Persson, M. E., Roth, L. S. V., Johnson, M., Wright, D., Jensen, P., Human-directed social behavior in dogs shows significant heritability. Genes Brain Behav. 14, 337-344 (2015).
19. vonHoldt, B. M., Pollinger, J. P., Lohmueller, K. E., Han, E., Parker, H. G., Quignon, P., Degenhardt, J. D., Boyko, A. R., Earl, D. A., Auton, A., Reynolds, A., Bryc, K., Brisbin, A., Knowles, J. C., Mosher, D. S., Spady, T. C., Elkahloun, A., Geffen, E., Pilot, M., Jedrzejewski, W., Greco, C., Randi, E., Bannasch, D., Wilton, A., Shearman, J., Musiani, M., Cargill, M., Jones, P. G., Qian, Z., Huang, W., Ding, Z.-L., Zhang, Y. P., Bustamante, C. D., Ostrander, E. A., Novembre, J., Wayne, R. K., Genome-wide SNP and haplotype analyses reveal a rich history underlying dog domestication. Nature 464, 898-902 (2010).
20. Schubert, C., The genomic basis of the Williams-Beuren syndrome. Cell. Mol. Life Sci. 66, 1178-1197 (2009).
21. Meyer-Lindenberg, A., Mervis, C. B., Berman, K. F., Neural mechanisms in Williams syndrome: a unique window to genetic influences on cognition and behaviour. Nat. Rev. Neurosci. 7, 380-393 (2006).
22. Jones, W., Bellugi, U., Lai, Z., Chiles, M., Reilly, J., Lincoln, A., Adolphs, R., II. Hypersociability in Williams syndrome. J. Cognitive Neurosci. 12, 30-46 (2000).
23. Ewart, A. K., Morris, C. A., Atkinson, D., Jin, W., Sternes, K., Spallone, P., Stock, A. D., Leppert, M., Hemizygosity at the elastin locus in a developmental disorder, Williams syndrome. Nat. Genet. 5, 11-16 (1993).
24. Wan, M., Hejjas, K., Ronai, Z., Elek, Z., Sasvari-Szekely, M., Champagne, F. A., Miklósi, Á., Kubinyi, E., DrD4 and TH gene polymorphisms are associated with activity, impulsivity and inattention in Siberian Husky dogs. Anim. Genet. 44, 717-727 (2013).
25. Kis, A., Bence, M., Lakatos, G., Pergel, E., Turcsan, B., Pluijmakers, J., Vas, J., Elek, Z., Bruder, I., Foldi, L., Sasvari-Szekely, M., Miklósi, A., Ronai, Z., Kubinyi, E., Oxytocin receptor gene polymorphisms are associated with human directed social behavior in dogs (Canis familiaris). PLoS One 9(1): e83993. doi:10.1371/journal.pone.0083993 (2014).
26. Jakovcevic, A., Mustaca, A., Bentosela, M., Do more sociable dogs gaze longer to the human face than less sociable ones? Behav. Process. 90, 217-222 (2012).
27. Bentosela, M., Wynne, C. D., D'Orazio, M., Elgier, A., Udell, M. A., Sociability and gazing toward humans in dogs and wolves: Simple behaviors with broad implications. J. Exp. Anal. Behav. 105, 68-75 (2016).
28. Brubaker, L., Dasgupta, S., Bhattacharjee, D., Bhadra, A., Udell, M. A. R., Differences in problem-solving between canid populations: Do domestication and lifetime experience affect persistence? Anim. Cogn. https://doi.org/10.1007/s10071-017-1093-7 (2017).
29. Walsh, T., McClellan, J. M., McCarthy, S. E., Addington, A. M., Pierce, S. B., Cooper, G. M., Nord, A. S., Kusenda, M., Malhotra, D., Bhandari, A., Stray, S. M., Rippey, C. F., Roccanova, P., Makarov, V., Lakshmi, B., Findling, R. L., Sikich, L., Stromberg, T., Merriman, B., Gogtay, N., Butler, P., Eckstrand, K., Noory, L., Gochman, P., Long, R., Chen, Z., Davis, S., Baker, C., Eichler, E. E., Meltzer, P. S., Nelson, S. F., Rare structural variants disrupt multiple genes in neurodevelopmental pathways in schizophrenia. Science 320, 539-543 (2008).
30. Cuscó, I., Corominas, R., Bayés, M., Flores, R., Rivera-Brugués, N., Campuzano, V., Perez-Jurado, L. A., Copy number variation at the 7q11. 23 segmental duplications is a susceptibility factor for the Williams-Beuren syndrome deletion. Genome Res. 18, 683-694 (2008).
31. Wong, K., Keane, T. M., Stalker, J., Adams, D. J., Enhanced structural variant and breakpoint detection using SVMerge by integration of multiple detection methods and local assembly. Genome Biol. 11, R128 (2010).
32. Hart, S. N., Sarangi, V., Moore, R., Baheti, S., Bhavsar, J. D., Couch, F. J., Koher, J.-P. A., SoftSearch: integration of multiple sequence features to identify breakpoints of structural variations. PLoS One 8, e83356 (2013).
33. Qi, J., Zhao, F., inGAP-sv: a novel scheme to identify and visualize structural variation from paired end mapping data. Nucleic Acids Res. 39, W567-W575 (2011).
34. Korenberg, J. R., Chen, X.-N., Hirota, H., VI. Genome structure and cognitive map of Williams Syndrome. J. Cognitive Neuroci. 12(1), 89-107 (2000).
35. Young, E. J., Lipina, T., Tam, E., Mandel, A., S. Clapcote, J., Bechard, A. R., Chambers, J., Mount, H. T. J., Fletcher, P. J., Roder, J. C., Osborne, L. R., Reduced fear and aggression and altered serotonin metabolism in Gtf2ird1-tagged mice. Genes Brain Behav. 7, 224-234 (2008).
36. Li, H. H., Roy, M., Kuscuoglu, U., Spencer, C. M., Halm, B., Harrison, K. C., Bayle, J. H., Splendore, A., Ding, F., Meltzer, L. A., Wright, E., Paylor, R., Deisseroth, K., Francke, U., Induced chromosome deletions cause hypersociability and other features of Williams-Beuren syndrome in mice. Mol. Med 1, 50-65 (2009).
37. Doyle, T. F., Bellugi, U., Korenberg, J. R., Graham, J., “Everybody in the world is my friend” hypersociability in young children with Williams syndrome. Am. J. Med Genet. A 124, 263-273 (2004).
38. Edelmann, L., Prosnitz, A., Pardo, S., Bhatt, J., Cohen, N., Lauriat, T., Ouchanov, L., Gonzalez, P. J., Manghi, E. R., Bondy, P., Esquivel, M., Monge, S., Delgado, M. F., Splendore, A., Francke, U., Burton, B. K., McInnes, L. A., An atypical deletion of the Williams-Beuren syndrome interval implicates genes associated with defective visuospatial processing and autism. J. Med Genet. 44, 136-143 (2007).
39. McLaren, W., Pritchard, B., Rios, D., Chen, Y., Flicek, P., Cunningham, F., Deriving the consequences of genomic variants with the Ensembl API and SNP Effect Predictor. Bioinformatics 26, 2069-2070 (2010).
40. Hubbard, T., Barker, D., Birney, E., Cameron, G., Chen, Y., Clark, L., Cox, T., Cuff, J., Curwen, V., Down, T., Durbin, R., Eyras, E., Gilbert, J., M Hammond, Huminiecki, L., Kasprzyk, A., Lehvaslaiho, H., Lijnzaad, P., Melsopp, C., Mongin, E., Pettett, R., Pocock, M., Potter, S., Rust, A., Schmidt, E., Searle, S., Slater, G., Smith, J., Spooner, W., Stabenau, A., Stalker, J., Stupka, E., Ureta-Vidal, A., Vastrik, I., Clamp, M., The Ensembl genome database project. Nucleic Acids Res. 30, 38-41 (2002).
41. American Kennel Club, The New Complete Dog Book: Official Breed Standards and All-New Profiles for 200 Breeds (21^{st} Edition). Irvine, Calif.: Lumina Media (2014).
42. Bayes, M., Magano, L. F., Rivera, N., Flores, R., Perez Jurado, L. A., Mutational mechanisms of Williams-Beuren syndrome deletions. Am. J. Hum. Genet. 73, 131-151 (2003).
43. Reymond, A., Henrichsen, C. N., Harewood, L., Merla, G., Side effects of genome structural changes. Curr. Opin. Genet. Dev. 17, 381-386 (2007).
44. Merla, G., Micale, L., Fusco, C., Loviglio, M. N., Molecular Genetics of Williams-Beuren Syndrome. In: eLS. John Wiley & Sons, Ltd: Chichester (2012).
45. Bayarsaihan, D., Ruddle, F. H., Isolation and characterization of BEN, a member of the TFII-I family of DNA-binding proteins containing distinct helix-loop-helix domains. Proc. Natl. Acad. Sci. USA 97(13), 7342-7347 (2000).
46. Tipney, H. J., Hinsley, T. A., Brass, A., Metcalfe, K., Donnai, D., Tassabehji, M., Isolation and characterization of GTF2IRD2, a novel fusion gene and member of the TFII-I family of transcription factors, deleted in William-Beuren syndrome. Eur. J. Hum. Genet. 12, 551-560 (2004).
47. Tassabehji, M., Hammond, P., Karmiloff-Smith, A., Thompson, P., Thorgeirsson, S. S., Durkin, M. E., Popescu, N.C., Hutton, T., Metcalfe, K., Rucka, A., Stewart, H., Read, A. P., Maconochie, M., Donnai, D., GTF2IRD1 in craniofacial development of humans and mice. Science 310(5751), 1184-1187 (2005).
48. Chimge, N. O., Makeyev, A. V., Ruddle, F. H., Bayarsaihan, D., Identification of the TFII-I family target genes in the vertebrate genome. Proc. Natl. Acad. Sci. USA 105(26), 9006-9010 (2008).
49. Porter, M. A., Dobson-Stone, C., Kwok, J. B., Schofield, P. R., Beckett, W., Tassabehji, M., A role for transcription factor GTF2IRD2 in executive function in Williams-Beuren Syndrome. PLoS One 7(10), e47457 (2012).
50. Sakurai, T., Dorr, N. P., Takahash., N., McInnes, L. A., Elder, G. A., Buxbaum, J. D., Haploinsufficiency of Gtf2i, a gene deleted in Williams Syndrome, leads to increases in social interactions. Autism Res. 4, 28-39 (2011).
51. Procyshyn, T. L., Spence, J., Read, S., Watson, N. V., Crespi, B. J., The Williams syndrome prosociality gene GTF2I mediates oxytocin reactivity and social anxiety in a healthy population. Biol. Letters 13(4), http://dx.doi.org/10.1098/rsbl.2017.0051 (2017).
52. Merla, G., Howald, C., Henrichsen, C. N., Lyle, R., Wyss, C., Zabot, M. T., Antonarakis, S. E., Reymond, A., Submicroscopic deletion in patients with Williams-Beuren syndrome influences expression levels of the nonhemizygous flanking genes. Am. J. Hum. Genet. 79(2), 332-341 (2006).
53. Li, H. H., Roy, M., Kuscuoglu, U., Spencer, C. M., Halm, B., Harrison, K. C., Joseph H Bayle, Alessandra Splendore, Feng Ding, Leslie A Meltzer, Elena Wright, Richard Paylor, Karl Deisseroth, and Uta Francke. Induced chromosome deletions cause hypersociability and other features of Williams-Beuren syndrome in mice. Embo Mol. Med. 1(1), 50-65 (2009).
54. Lau, K. S., Khan, S., Dennis, J. W., Genome-scale identification of UDP-GlcNAc-dependent pathways. Proteomics 8(16), 3294-3302 (2008).
55. Axelsson, E., Ratnakumar, A., Arendt, M.-L., Maqbool, K., Webster, M. T., Perloski, M., Liberg, O., Arnemo, J. M., Hedhammar, A., Lindblad-Toh, K., The genomic signature of dog domestication reveals adaptation to a starch-rich diet. Nature 495(7441), 360-364 (2013).
56. Cowley, M., Oakey, R. J., Transposable elements re-wire and fine-tune the transcriptome. PLOS Genet. 9(1), e1003234 (2013).
57. Wang, W., Kirkness, E. F., Short interspersed elements (SINEs) are a major source of canine genomic diversity. Genome Res. 15, 1798-1808 (2005).
58. Janowitz Koch, I., Clark, M. M., Thompson, M. J., Deere-Machemer, K. A., Wang, J., Duarte, L., Gnanadesikan, G. E., McCoy, E. L., Rubbi, L., Stahler, D. R., Pellegrini, M., Ostrander, E. A., Wayne, R. K., Sinsheimer, J. S, vonHoldt, B. M., The concerted impact of domestication and transposon insertions on methylation patterns between dogs and grey wolves. Mol. Ecol. 25(8), 1838-1855 (2016)
59. Lin, L., Faraco, J., Li, R. Kadotani, H., Rogers, W., Lin, X., Qiu, X., de Jong, P. J., Nishino, S., Mignot, E., The sleep disorder canine narcolepsy is caused by a mutation in the hypocretin (orexin) receptor 2 gene. Cell 98, 365-376 (1999).
60. Pele, M., Tiret, L., Kessler, J. L., Blot, S., Panthier, J. J., SINE exonic insertion in the PTPLA gene leads to multiple splicing defects and segregates with the autosomal recessive centronuclear myopathy in dogs. Hum. Mol. Genet. 14, 1417-1427 (2005).
61. Clark, L. A., Wahl, J. M., Rees, C. A., Murphy, K. E., Retrotransposon insertion in SILV is responsible for merle patterning of the domestic dog. Proc. Natl. Acad. Sci. USA 103, 1376-1381 (2006).
62. Sutter, N. B., Bustamante, C. D., Chase, K., Gray, M. M., Zhao, K., Zhu, L., Padhukasahasram, B., Karlins, E., Davis, S., Jones, P. G., Quignon, P., Johnson, G. S., Parker, H. G., Fretwell, N., Mosher, D. S., Lawler, D. F., Satyaraj, E., Nordborg, M., Lark, K. G., Wayne, R. K., Ostrander, E. A., A single IGF1 allele is a major determinant of small size in dogs. Science 316(5821), 112-115 (2007).
63. Parker, H. G., vonHoldt, B. M., Quignon, P., Margulies, E. H., Shao, S., Mosher, D. S., Spady, T. C., Elkahloun, A., Cargill, M., Jones, P. G., Maslen, C. L. Acland, G. M., Sutter, N. B., Kuroki, K., Bustamante, C. D., Wayne, R. K., Ostrander, E. A., An expressed fgf4 retrogene is associated with breed-defining chondrodysplasia in domestic dogs. Science 325(5943), 995-998 (2009).
64. Gray, M. M. Sutter, N. B., Ostrander, E. A., Wayne, R. K., The IGF1 small dog haplotype is derived from Middle Eastern grey wolves. BMC Biology 8, 16 (2010).
65. Karlsson, E. K., Lindblad-Toh, K., Leader of the pack: gene mapping in dogs and other model organisms. Nat. Rev. Genet. 9, 713-725 (2008).
66. Boyko, A. R., The domestic dog: man's best friend in the genomic era. Genome Biology 12, 216 (2011).
67. Anderson, T. M., vonHoldt, B. M., Candille, S. I., Musiani, M., Greco, C., Stahler, D. R., Smith, D. W., Padhukasahasram, B., Randi, E., Leonard, J. A., Bustamante, C. D., Ostrander, E. A., Tang, H., Wayne, R. K., Barsh, G. S., Molecular and evolutionary history of melanism in North American gray wolves. Science 323(5919), 1339-1343 (2009).
68. Frank, H., Frank, M. G., On the effects of domestication on canine social development and behavior. Appl. Anim. Ethol. 8, 507-525 (1982).
69. Udell, M. A. R., Dorey, N. R., Wynne, C. D. L., The performance of stray dogs (Canis familiaris) living in a shelter on human-guided object-choice tasks. Anim. Behav. 79, 717-725 (2010).
70. Klinghammer, E., Goodman, P., Socialization and management of wolves in captivity. In H. Frank (Ed.), Man and Wolf: Advances, Issues, and Problems in Captive Wolf Research. Springer (1987).
71. Udell, M. A., Dorey, N. R., Wynne, C. D., Wolves outperform dogs in following human social cues. Anim. Behav. 76, 1767-1773 (2008).
72. McHugh, M. L., Interrater reliability: the kappa statistic. Biochem. medica 22, 276-282 (2012).
73. Udell, M. A. R., Dorey, N. R., Wynne, C. D. L., Wolves outperform dogs in following human social cues. Anim. Behav. 76, 1767-1773 (2008).
74. Raiche, G., nFactors: An R package for parallel analysis and non graphical solutions to the Cattell scree test. R package version 2 (2010).
75. Aschard, H., Vilhjálmsson, B. J., Greliche, N., Morange, P. E., Tregouet, D. A., Maximizing the power of principal-component analysis of correlated phenotypes in genome-wide association studies. Am. J. Hum. Genet. 94, 662-676 (2014).
76. Cunningham, F., Amode, M. R., Barrell, D., Beal, K., Billis, K., Brent, S., Carvalho-Silva, D., Clapham, P., Coates, G., Fitzgerald, S., Gil, L., C. Girón, Garcí., Gordon, L., Hourlier, T., Hunt, S. E., Janacek, S. H., Johnson, N., Juettemann, T., Kähäri, A. K., Keenan, S., Martin, F. J., Maurel, T., McLaren, W., Murphy, D. N., Nag, R., Overduin, B., Parker, A., Patricio, M., Perry, E., Pignatelli, M., Riat, H. S., Sheppard, D., Taylor, K., Thormann, A., Vullo, A., Wilder, S. P., Zadissa, A., Aken, B. L., Birney, E., Harrow, J., Kinsella, R., Muffato, M., Ruffier, M., Searle, S. M. J., Spudich, G., Trevanion, S. J., Yates, A., Zerbino, D. R., Flicek, P., Ensembl 2015. Nucleic Acids Res. 43, D662-D669 (2015).
77. Martin, M., Cutadapt removes adapter sequences from high-throughput sequencing reads. EMBnet Journal 17, pp. 10-12 (2011).
78. Li, H., Durbin, R., Fast and accurate short read alignment with Burrows-Wheeler Transform. Bioinformatics 25, 1754-1760 (2009).
79. Li, H., Handsaker, B., Wysoker, A., Fennell, T., Ruan, J., Homer, N., Marth, G., Abecasis, G., Durbin, R., 1000 Geome Project Data Processing Subgroup, The sequence alignment/map format and SAMtools. Bioinformatics 25, 2078-2079 (2009).
80. Korneliussen, T. S., Albrechtsen, A., Nielsen, R., ANGSD: analysis of next generation sequencing data. BMC Bioinformatics 15, 356 (2014).
81. Delaneau, O., Marchini, J., Zagury, J.-F., A linear complexity phasing method for thousands of genomes. Nat. Methods 9, 179-181 (2012).
82. Sabeti, P. C., Varilly, P., Fry, B., Lohmueller, J., Hostetter, E., Cotsapas, C., Xie, X., Byrne, E. H., McCarroll, S. A., Gaudet, R., Schaffner, S. F., Lander, E. S., The International HapMap Consortium, Genome-wide detection and characterization of positive selection in human populations. Nature 449, 913-918 (2007).
83. Chen, K., Wallis, J. W., McLellan, M. D., Larson, D. E., Kalicki, J., Pohl, C. S., McGrath, S. D., Wendl, M. C., Zhang, Q., Locke, D. P., Shi, X., Fulton, R. S., Ley, T. J., Wilson, R. K., Ding, L., Mardis, E. R., BreakDancer: an algorithm for high-resolution mapping of genomic structural variation. Nat. Methods 6, 677-681 (2009).
84. Ye, K., Schulz, M. H., Long, Q., Apweiler, R., Ning, Z., Pindel: a pattern growth approach to detect break points of large deletions and medium sized insertions from paired-end short reads. Bioinformatics 25, 2865-2871 (2009).
85. Wong, K., Keane, T. M., Stalker, J., Adams, D. J., Enhanced structural variant and breakpoint detection using SVMerge by integration of multiple detection methods and local assembly. Genome Biol. 11, R128 (2010).
86. Hart, S. N., Sarangi, V., Moore, R., Baheti, S., Bhavsar, J. D., SoftSearch: integration of multiple sequence features to identify breakpoints of structural variations. PLoS One 8, e83356 (2013).
87. Tattini, L., D'Aurizio, R., Magi, A., Detection of genomic structural variants from next-generation sequencing data. Frontiers Bioeng. Biotechnol. 3 (2015).
88. Qi, J., Zhao, F., inGAP-sv: a novel scheme to identify and visualize structural variation from paired end mapping data. Nucleic Acids Res. 39, W567-W575 (2011).
89. Hollox, E. J., “The challenges of studying complex and dynamic regions of the human genome” in Genomic Structural Variants (Spring, New York), pp. 187-207 (2012).
90. Quinlan, A. R., Hall I. M., Characterizing complex structural variation in germline and somatic genomes. Trends Genet. 28, 43-53 (2012).
91. Kent, W. J., Sugnet, C. W., Furey, T. S., Roskin, K. M., Pringle, T. H., Zahler, A. M., Haussler, D., The human genome browser at UCSC. Genome Res. 12, 996-1006 (2002).
92. Decker B., Davis, B. W., Rimbault, M., Long, A. H., Karlins, E., Jagannathan, V., Reiman, R., Parker, H. G., Drögmüeller, C., Corneveaux, J. J., Chapman, E. S., Trent, J. M., Leeb, T., Huentelman, M. J., Wayne, R. K., Karyali, D. M., Ostrander, E. A., Comparison against 186 canid whole-genome sequences reveals survival strategies of an ancient clonally transmissible canine tumor. Genome Res. 25, 1646-1655 (2015).
93. Zhou, X., Stephens, M., Genome-wide efficient mixed-model analysis for association studies. Nat. Genet. 44, 821-824 (2012).
94. Stich, B., Möhring, J., Piepho, H.-P., Heckenberger, M., Buckler, E. D., Melchinger, A. E., Comparison of mixed-model approaches for association mapping. Genetics 178, 1745-1754 (2008).
95. Mandel, J. R., Nambeesan, S., Bowers, J. E., Marek, L. F., Ebert, D., Rieseberg, L. H., Knapp, S. J., Burke, J., Association mapping and the genomic consequences of selection in sunflower. PLoS Genet. 9, e1003378 (2013).
96. Tabangin, M. E., Woo, J. G., Martin, L. J., The effect of minor allele frequency on the likelihood of obtaining false positives. BMC Proceedings 3, S41 (2009).
97. Gao, X., Starmer, J., Martin, E. R., A multiple testing correction method for genetic association studies using correlated single nucleotide polymorphisms. Genet. Epidemiol. 32, 361-369 (2008).
98. Purcell, S., Neale, B., Todd-Brown, K., Thomas, L., Ferreira, M. A., Bender, D., Maller, J., Sklar, P., P. I. Bakker, d., Daly, M. J., Sham, P. C., PLINK: a tool set for whole-genome association and population-based linkage analyses. Am. J. Hum. Genet. 81, 559-575 (2007).
99. Untergasser, A., Cutcutache, I., Koressaar, T., Ye, J., Faircloth, B. C., Remm, M., Rozen, S. G., Primer3—New capabilities and interfaces. Nucleic Acids Res. 40(15), e115 (2012).
100. Zou, Q., Wang, X., Liu, Y., Ouyang, Z., Long, H., Wei, S., Xin, J., Zhao, B., Lai, S., Shen, J., Ni, Q., Yang, H., Zhong, H., Li, L., Hu, M., Zhang, Q., Zhou, Z., He, J., Yan, Q., Fan, N., Zhao, Y., Liu, Z., Guo, L., Huang, J., Zhang, G., Ying, J., Lai, L., Gao, X., Generation of gene-target dogs using CRISPR/Cas9 system. J. Mol. Cell. Biol. 7(6), 580-583 (2015).
101. Abraham, G., Inouye, M., Fast principal component analysis of large-scale genome-wide data. PLoS One 9, e93766 (2014).
102. Zhang, B., Kirov, S. A., Snoddy, J. R., WebGestalt: an integrated system for exploring gene sets in various biological contexts. Nucleic Acids Res. 33 (Web Server Issue), W741-748 (2005).
103. Wang, J., Duncan, D., Shi, Z., Zhang, B., WEB-based GEne SeT AnaLysis Toolkit (WebGestalt): update 2013. Nucleic Acids Res. 41 (Web Server Issue), W77-83 (2013).
104. Benjamini, Y., Hochberg, Y., Controlling the false discovery rate: a practical and powerful approach to multiple testing. J. R. Stat. Soc. Ser. BMet. 57, 289-300 (1995).
105. Fusco, C., Micale, L., Augello, B., Pellico, T., Menghini, D., Alferi, P., Digilio, M. C., Mandriani, B., Carella, M., Palumbo, O., Vicari, S., Merla, G., Smaller and larger deletions of the Williams Beuren syndrome region implicate genes involved in mild facial phenotype, epilepsy and autistic traits. Eur. J. Hum. Genet. 22, 64-70 (2014).

1. A method for predicting the probability of a canine exhibiting a sociable behavior comprising:
(a) genotyping a biological sample from a canine;
(b) counting the number of structural variants within the Williams-Beuren Syndrome (WBS) locus on canine chromosome 6; and
(c) predicting the probability of the canine exhibiting a sociable behavior based on the number of structural variants.

2. A method of ranking dogs or wolves according to their likely level of exhibiting a sociable behavior comprising:
(a) obtaining a biological sample from a first dog or wolf;
(b) determining the number of structural variants within the Williams-Beuren Syndrome (WBS) locus on chromosome 6 of the first dog or wolf;
(c) obtaining a biological sample from a second dog or wolf;
(d) determining the number of structural variants within the Williams-Beuren Syndrome (WBS) locus on chromosome 6 of the second dog or wolf; and
(e) ranking the first dog as being more likely to exhibit a sociable behavior than the second dog if the number of structural variants determined in step (b) is greater than the number of structural variants determined in step (d); or
(f) ranking the second dog as being more likely to exhibit a sociable behavior than the first dog if the number of structural variants determined in step (d) is greater than the number of structural variants determined in step (b).

3. The method of claim 2 wherein the biological sample is blood, saliva, cerebrospinal fluid, skin, or urine.

4. The method of claim 2 wherein genotyping the biological sample includes PCR amplification and agarose gel electrophoresis.

5. The method of claim 2 wherein genotyping the biological sample utilizes at least one primer selected from the group consisting of:
(SEQ ID NO: 1)
CCCCTTCAGCCAGCATATAA,
(SEQ ID NO: 2)
TTCTCTGGGCTGTCTGGACT,
(SEQ ID NO: 3)
AAGTTTCTCTGATGGAAAACACA,
(SEQ ID NO: 4)
GGTGGCTGGAAATTTCAGTAG,
(SEQ ID NO: 5)
TGGAGCCATGATTAGGAAGG,
(SEQ ID NO: 6)
TAAGGAAGGACCCCATTTCC,
(SEQ ID NO: 7)
TGCTGCTTCATGTTCTGTGA,
(SEQ ID NO: 8)
TGGTGCATTAGCTTTGGTTG,
(SEQ ID NO: 9)
AACCACAGGAACAAAACCTCA,
and
(SEQ ID NO: 10)
CCTCCTGTTGGACATTTGGA.

6. The method of claim 2 wherein the structural variants are transposable elements that interrupt a gene in the WBS locus.

7. The method of claim 6 wherein the transposable elements are retrotransposons.

8. The method of claim 7 wherein the retrotransposons are short interspersed nuclear elements (SINEs) or a long interspersed nuclear elements (LINEs).

9. The method of claim 2 wherein at least one structural variant occurs within at least one gene selected from the group consisting of GTF2I, GTF2IRD1, and WBSCR17.

10. The method of claim 2 wherein the social behavior is selected from the group consisting of attentional bias to social stimuli (ABS), hyper-sociability (HYP), and social interest in strangers (SIS).

11. The method of claim 2 wherein at least one structural variant is found at Cfa6.6, Cfa6.7, Cfa6.66, or Cfa6.83.

12. A method of screening a dog or wolf library comprising:
(a) obtaining a genomic library from a dog or wolf that contains the Williams-Beuren Syndrome (WBS) locus on canine chromosome 6;
(b) determining the number of structural variants in the WBS locus.

13. The method of claim 12 wherein the locations of the structural variants are also determined.

14. The method of claim 12 wherein step (b) comprises determining the number of structural variants in at least one of GTF2I, GTF2IRD1, and WBSCR17.

15. The method of claim 12 wherein step (b) comprises determining the number of structural variants in all of GTF2I, GTF2IRD1, and WBSCR17.

16. The method of claim 12 wherein step (b) comprises the use of the polymerase chain reaction (PCR) to amplify at least one DNA fragment from the WBS locus.

17. The method of claim 16 wherein the DNA fragment comprises at least one of the loci Cfa6.6, Cfa6.7, Cfa6.66, or Cfa6.83.

18. The method of claim 12 wherein step (b) comprises the use of PCR to amplify the locus Cfa6.6 using the primers CCCCTTCAGCCAGCATATAA (SEQ ID NO: 1) (forward) and TTCTCTGGGCTGTCTGGACT (SEQ ID NO: 2) (reverse).

19. The method of claim 12 wherein step (b) comprises the use of PCR to amplify the locus Cfa6.6 using the primers AAGTTTCTCTGATGGAAAACACA (SEQ ID NO: 3) (forward) and GGTGGCTGGAAATTTCAGTAG (SEQ ID NO: 4) (reverse).

20. The method of claim 12 wherein step (b) comprises the use of PCR to amplify the locus Cfa6.7 using the primers TGGAGCCATGATTAGGAAGG (SEQ ID NO: 5) (forward) and TAAGGAAGGACCCCATTTCC (SEQ ID NO: 6) (reverse).

21. The method of claim 12 wherein step (b) comprises the use of PCR to amplify the locus Cfa6.66 using the primers TGCTGCTTCATGTTCTGTGA (SEQ ID NO: 7) (forward) and TGGTGCATTAGCTTTGGTTG (SEQ ID NO: 8) (reverse).

22. The method of claim 12 wherein step (b) comprises the use of PCR to amplify the locus Cfa6.83 using the primers AACCACAGGAACAAAACCTCA (SEQ ID NO: 9) (forward) and CCTCCTGTTGGACATTTGGA (SEQ ID NO: 10) (reverse).

23. The method of claim 12 wherein step (b) comprises the use of agarose gel electrophoresis to identify DNA fragments from the WBS locus that have altered mobility compared to the corresponding fragments from the dog reference genome and that are indicative of structural variants in the WBS locus from the library.

24. The method of claim 12 wherein step (b) comprises a hybridization step using at least one probe from the WBS locus that identifies structural variants in the WBS locus. In some embodiments, the hybridization step comprises fluorescence in-situ hybridization (FISH).

25. A method of producing dogs that are more likely to exhibit a sociable behavior comprising:
(a) selecting a male and female dog for breeding that each are known to have at least one structural variant within Cfa6.6, Cfa6.7, Cfa6.66, or Cfa6.83 in the Williams-Beuren Syndrome (WBS) locus; and
(b) mating the dogs of step (a) to produce offspring.

26. The method of claim 25 wherein the male and female dogs are genotyped for the presence of structural variants within the Williams-Beuren Syndrome (WBS) locus.

27. The method of claim 25 wherein the at least one structural variant occurs within at least one gene selected from the group consisting of GTF2I, GTF2IRD1, and WBSCR17.

28. A method of editing the genome of a dog comprising:
(a) obtaining a dog;
(b) using clustered regularly interspaced short palindromic repeats (CRISPRs)/CRISPR-associated (Cas) 9 to inactivate a gene in the Williams-Beuren Syndrome (WBS) locus on canine chromosome 6.

29. The method of claim 28 wherein the gene is GTF2I, GTF2IRD1, or WBSCR17.

30. A kit for detecting the presence of structural variants within the Williams-Beuren Syndrome (WBS) locus of canines comprising one or more primers selected from the group consisting of:
(SEQ ID NO: 1)
CCCCTTCAGCCAGCATATAA,
(SEQ ID NO: 2)
TTCTCTGGGCTGTCTGGACT,
(SEQ ID NO: 3)
AAGTTTCTCTGATGGAAAACACA,
(SEQ ID NO: 4)
GGTGGCTGGAAATTTCAGTAG,
(SEQ ID NO: 5)
TGGAGCCATGATTAGGAAGG,
(SEQ ID NO: 6)
TAAGGAAGGACCCCATTTCC,
(SEQ ID NO: 7)
TGCTGCTTCATGTTCTGTGA,
(SEQ ID NO: 8)
TGGTGCATTAGCTTTGGTTG,
(SEQ ID NO: 9)
AACCACAGGAACAAAACCTCA,
and
(SEQ ID NO: 10)
CCTCCTGTTGGACATTTGGA.

31. The kit of claim 30 wherein the kit comprises the primers CCCCTTCAGCCAGCATATAA (SEQ ID NO: 1) and TTCTCTGGGCTGTCTGGACT (SEQ ID NO: 2).

32. The kit of claim 30 wherein the kit comprises the primers AAGTTTCTCTGATGGAAAACACA (SEQ ID NO: 3) and GGTGGCTGGAAATTTCAGTAG (SEQ ID NO: 4).

33. The kit of claim 30 wherein the kit comprises the primers TGGAGCCATGATTAGGAAGG (SEQ ID NO: 5) and TAAGGAAGGACCCCATTTCC (SEQ ID NO: 6).

34. The kit of claim 30 wherein the kit comprises the primers TGCTGCTTCATGTTCTGTGA (SEQ ID NO: 7) and TGGTGCATTAGCTTTGGTTG (SEQ ID NO: 8).

35. The kit of claim 30 wherein the kit comprises the primers AACCACAGGAACAAAACCTCA (SEQ ID NO: 9) and CCTCCTGTTGGACATTTGGA (SEQ ID NO: 10).

36. The kit of claim 30 further comprising instructions for use.

37. The kit of claim 30 wherein the primers are labeled using a detectable marker.

38. The kit of claim 30 further comprising at least one of a buffer, dNTPs, a DNA polymerase, a DNA ligase, or a restriction enzyme.
